SEQUENCE LISTING (6 sequences)

SEQ ID NO: 1 - DNA, 23 bp, Artificial Sequence
  synthetic - gRNA A SLA I target
ccaggaccagagccaggacatgg

SEQ ID NO: 2 - DNA, 23 bp, Artificial Sequence
  synthetic - gRNA B SLA I target
gagaccaggccctcaggggatgg

SEQ ID NO: 3 - DNA, 23 bp, Artificial Sequence
  synthetic - gRNA C SLA target
ccagaagtgggcggccctggtgg

SEQ ID NO: 4 - DNA, 5821 bp, Artificial Sequence
  synthetic - HLA A2 vector
  misc_feature (613)..(667): SLA 5′ homology arm
  misc_feature (685)..(768): PHi Recombinase ATTP site
  misc_feature (780)..(1806): hygromycin resistance gene
  misc_feature (1821)..(2047): SV40 poly A tail
  misc_feature (2079)..(2163): Phi Recombinase ATTB site
  misc_feature (2175)..(3273): HLA-A2 ORF
  misc_feature (3274)..(3501): BGH polyA
  misc_feature (3515)..(3734): SLA-3′ homology arm
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcggcctgcgcaacacgcgatcgcggcgcgccaccatggaccatggtaatgatagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagccaccatggaatcgatataagctctccacccacccggctctgctcagcttctccccagaccccgaggctgaggatcatcgatcttaagagaagcggttttcgggagtagtgccccaactggggtaacctttgagttctctcagttgggggcgtagggtcgccgacatgacaccttaaggatatcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgccatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgaacctgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgggtcgacgatatcgtttaaacacgcgttctagatcacggtctcgaagccgcggtgcgggtgccagggcgtgcccttgggctccccgggcgcgtactccacctcacccatctggtccatctagattcgaaatggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaattaattaaggactgagggacacaagatttgttcacatcccactttgtgacttcagatcccctgactcctatttctgcagctgcctctgaaagtgtctgtgttcctatgagcatcctgagaggaggtggggaccctggcccagccctgccccccacgtcccctcctcaccctgacctgtgttctcttccctgatcctctttccagttcttgcaagtgggagctgggctttaattaatgcgcagcgatcgcggccgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 23 bp, Artificial Sequence
  synthetic - gRNA 5′ SLA
ccccgaggctgaggatcatgggg

SEQ ID NO: 6 - DNA, 23 bp, Artificial Sequence
  synthetic - gRNA 3′ SLA
aactgccttgtggggactgaggg

(19) United States
(12) Patent Application Publication
(10) Pub. No.: US 2018/0184630 A1
(43) Pub. Date: Jul. 5, 2018

(54) TRANSGENIC PIGS WITH GENETIC MODIFICATIONS OF SLA

(71) Applicant: INDIANA UNIVERSITY RESEARCH & TECHNOLOGY CORPORATION, Indianapolis, IN (US)

(72) Inventor: A. Joseph Tector, III, Carmel, IN (US)

(21) Appl. No.: 15/739,469

(22) PCT Filed: Jun. 24, 2016
(86) PCT No.: PCT/US16/39279

§ 371 Date: Dec. 22, 2017

Related U.S. Application Data
(60) Provisional application No. 62/184,996, filed on Jun. 26, 2015.
(60) Provisional application No. 62/301,777, filed on Mar. 1, 2016.

(51) Int. Cl. A01K 67/027; A61K 35/407; C07K 14/705; C12N 15/877; C12N 15/85; A61K 35/44

(52) U.S. Cl. CPC A01K 67/0275; A61K 35/407; C07K 14/705; C12N 15/877; A01K 2267/025; A61K 35/44; A01K 2207/15; A01K 2217/075; A01K 2227/108; C12N 15/8509

(57) ABSTRACT

The application provides methods of improving a rejection related symptom, reducing premature separation and methods of producing a compound of interest with an altered epitope profile are provided. Transgenic pigs with a disrupted gene or genes, and porcine organs, tissues, and cells therefrom are provided.

### CROSS-REFERENCE TO RELATED APPLICATIONS

This application claims the benefit of U.S. Provisional Application Ser. Nos. 62/184,996, filed Jun. 26, 2015, and 62/301,777, filed Mar. 1, 2016, each of which is incorporated by reference herein as if set forth in its entirety.

### INCORPORATION OF SEQUENCE LISTING

The sequence listing in text format submitted herewith is incorporated by reference in its entirety for all purposes.

### STATEMENT REGARDING FEDERALLY SPONSORED RESEARCH OR DEVELOPMENT

Not applicable.

### FIELD OF THE INVENTION

It is well known that transplants from one animal into another animal of the same species, such as human to human, are a routine treatment option for many serious conditions including kidney, heart, lung, liver and other organ disease and skin damage such as severe burn disease. However, it is well known that there are not enough suitable organs available for transplant to meet current or expected clinical demands for organ transplants. Approximately 100,000 patients are on the kidney transplant list, and they remain on the waiting list an average of nearly five years before receiving a transplant or dying. In patients with kidney failure, dialysis increases the length of time the patient can wait for a transplant. More than 18,000 patients are on the UNOS liver transplant national waiting list, yet less than 7,000 transplants are performed annually in the United States. There is no system comparable to dialysis available for patients with liver disease or liver failure.

Xenotransplantation, the transplant of organs, tissues or cells from one animal into another animal of a different species, such as the transplantation of a pig organ into a human recipient has the potential to reduce the shortage of organs available for transplant, potentially helping thousands of people worldwide. However, xenotransplantation using standard, unmodified pig tissue into a human or other primate is accompanied by rejection of the transplanted tissue. The rejection may be a cellular rejection (lymphocyte mediated) or humoral (antibody mediated) rejection including but not limited to hyperacute rejection, an acute rejection, a chronic rejection, may involve survival limiting thrombocytopenia coagulopathy and an acute humoral xenograft reaction (AHXR). While not being limited by mechanism, both humoral and cellular rejection processes may target MHC molecules. The human hyperacute rejection response to pig antibodies present on transplanted tissue is so strong that the transplant tissue is typically damaged by the human immune system within minutes or hours of transplant into the human. Furthermore, different rejection mechanisms may predominate in an organ-preferred manner. An acute or rapid humoral rejection may begin within minutes of transplant; an acute or rapid cellular rejection may begin within days of the transplant. Both humoral and cellular rejections may also have a slower or chronic rejection phase; the chronic phases may occur for years. See Demetris et al. 1998 “Antibody-mediated Rejection of Human Orthotopic Liver Allografts. A study of liver transplantation across ABO blood group barriers”, Am J. Pathol 132:489-502; Nakamura et al 1993 “Liver allograft rejection in sensitized recipients. Observations in a Clinically Relevant Small Animal Model” Am J. Pathol. 142:1383-91; Furuya et al 1992. “Preformed Lymphocytotoxic Antibodies: the Effects of Class, Titer and Specificity on Liver v Heart Allografts” Hepatology 16:1415-22; Tector et al 2001. “Rejection of Pig Liver Xenografts in Patients with Liver Failure: Implications for Xenotransplantation”, Liver Transpl pp. 82-9; herein incorporated by reference in their entirety. For example, early development of thrombocytopenic coagulopathy is a major factor in non-human primate recipient death following xeno-transplant of a pig liver. Yet, if antibody mediated xenograft rejection is prevented, non-human primate (NHP) recipients of pig kidneys do not develop significant thrombocytopenia nor exhibit clinical manifestations of coagulopathy. See for example Ekser et al. 2012 “Genetically Engineered Pig to Baboon Liver Xenotransplantation: Histopathology of Xenografts and Native Organs” PLoS ONE pp e29720; Knosalla et al 2009, “Renal and Cardiac Endothelial Heterogeneity Impact Acute Vascular Rejection in Pig to Baboon Xenotransplantation”, Am J Transplant 1006-16; Shimizu et al 2012. “Pathologic Characteristics of Transplanted Kidney Xenografts”, J. Am. Soc. Nephrology 225-35; herein incorporated by reference in their entirety.

Pig cells express α(1,3) galactosyltransferase (αGal) and cytidine monophosphate-N-acetylneuraminic acid hydroxylase (CMAH), which are not found in human cells. The αGal enzyme catalyzes the formation of galactose-α1,3-galactose (αGal) residues on glycoproteins. CMAH converts the sialic acid N-acetylneuraminic acid (Neu5Ac) to N-glycolylneuraminic acid (Neu5Gc). Antibodies to the Neu5Gc and αGal epitopes are present in human blood prior to implantation of the tissue, and are involved in the intense and immediate antibody mediated rejection of implanted tissue. Additionally pig cells express multiple swine leukocyte antigens (SLAs). Unlike humans, pigs constitutively express class I and class II SLA's on endothelial cells. SLAs and human leukocyte antigens (HLAs) share considerable sequence homology (Varela et al 2003 J. Am. Soc Nephrol 14:2677-2683). Porcine class 1 SLAs include antigens encoded by the SLA-1, SLA-2, SLA-3, SLA-4, SLA-5, SLA-9 and SLA-11 loci. Porcine class II SLA's include antigens encoded by the SLA-DQ and SLA-DR loci. Anti-HLA antibodies are present in human blood prior to implantation of porcine tissue and cross react with SLA antigens on porcine tissues. The antibodies are present in the patient's blood prior to implantation of the tissue, contributing to the intense and immediate rejection of the implanted tissue. SLA antigens may also be involved with the T-cell mediated immune response.

Many strategies have been employed to address the rejection response including removing the genes encoding α(1,3) galactosyltransferase and CMAH to prevent expression of the enzymes, modifying the genes encoding α(1,3) galactosyltransferase and CMAH to reduce or limit expression of the enzymes, or otherwise limit the rejection response. U.S. Pat. No. 7,795,493 to Phelps et al describes a method for the production of a pig that lacks any expression of functional αGal. For instance, U.S. Pat. No. 7,547,816 to Day et al, describes a knockout pig with decreased expression of α(1,3) galactosyltransferase as compared to wild-type pigs. Although the Day pigs may have decreased expression of α(1,3) galactosyltransferase, Neu5Gc antigenic epitopes remain present and glycolipids from the Day pigs have αGal antigenic epitopes. Unfortunately, while the GTKO pig may have reduced anti-α-Gal antibodies as a barrier to xenotransplantation, studies using GTKO cardiac and renal xenografts in baboons show that the GTKO organs still trigger an immunogenic response, resulting in rejection or damage to the transplanted organ. Baboons transplanted with GTKO kidneys and treated with two different immunosuppressive regimens died within 16 days of surgery. Chen et al concluded “genetic depletion of Gal antigens does not provide a major benefit in xenograft survival” (Chen et al., (2005) Nature Med 11(12):1295-1298. U.S. Pat. No. 7,560,538 to Koike et al and U.S. Pat. Nos. 7,166,378 and 8,034,330 to Zhu et al describe methods for making porcine organs for transplantation that are less likely to be subject to delayed xenograft rejection and hyperacute rejection, respectively. Basnet et al examined the cytotoxic response of human serum to CMAH−/− mouse cells. Basnet et al concluded “the anti-Neu5Gc Ab-mediated immune response may be significantly involved in graft loss in xenogeneic cell transplantation, but not in organ transplantation” (Basnet et al., 2010 Xenotransplantation 17 (6):440-448). Attempts to reduce the rejection response by adding multiple human proteins (human CD39, CD55, CD59 and fucosyltransferase) to Gal-knockout pigs had limited effect on extending kidney xenograft survival (LeBas-Bernardet et al 2011 Transplantation Proceedings 43:3426-30). Clearly progress in this field is critically dependent upon the development of genetically modified pigs.

Unfortunately, developing homozygous transgenic pigs is a slow process, requiring as long as three years using traditional methods of homologous recombination in fetal fibroblasts followed by somatic cell nuclear transfer (SCNT), and then breeding of heterozygous transgenic animals to yield a homozygous transgenic pig. The development of new transgenic pigs for xenotransplantation has been hampered by the lack of pluripotent stem cells, relying instead on the fetal fibroblast as the cell upon which genetic engineering was carried out. For instance, the production of the first live pigs lacking any functional expression of α(1,3) galactosyltransferase (GTKO) was first reported in 2003.

Thus there is a need in the art for an improved, simple, replicable, efficient and standardized method of producing multiple transgenic (SLA−, αGal−, Class I HLA+) and (αGal−, SLA−, CMAH−; Class I HLA+) pigs having reduced SLA and αGal epitopes and increased Class I HLA epitopes or reduced Neu5Gc, SLA and αGal epitopes and increased Class I HLA epitopes as a source of transplant material for organs, tissue and cells for human transplant recipients. There is a need in the art for an improved, simple, replicable, efficient and standardized method of producing multiple transgenic (αGal−, SLA−, CMAH−) pigs having reduced SLA, Neu5Gc and αGal epitopes as a source for transplant material for organs, tissues and cells for human transplant recipients.

### BRIEF SUMMARY

This disclosure relates generally to methods of making porcine organs, tissues or cells with reduced SLA and αGal expression and increased Class I HLA expression, reduced α(1,3)galactosyltransferase, CMAH and SLA expression and increased Class I HLA expression, and reduced α(1,3)galactosyltransferase, CMAH and SLA expression for transplantation into a human.

A transgenic pig comprising a disrupted SLA gene and αGal gene and further comprising a nucleotide sequence encoding a human leukocyte antigen (HLA) class I polypeptide in the nuclear genome of at least one cell is provided. Expression of SLA and αGal in the transgenic pig are decreased as compared to expression in a wild-type pig, while expression of a HLA polypeptide in the transgenic pig is increased as compared to expression in a wild-type pig. A porcine organ, tissue or cell obtained from the transgenic pig is provided. A porcine organ, tissue or cell may be selected from the group consisting of skin, heart, liver, kidneys, lung, pancreas, thyroid, small bowel and components thereof. In an aspect, when tissue from the transgenic pig is transplanted into a human, a rejection related symptom is improved as compared to when tissue from a wild-type pig is transplanted into a human. Rejection related symptoms may occur as a result of cellular or humoral rejection responses. Such rejection responses may be acute or chronic. Cellular rejection responses are lymphocyte mediated; humoral rejection responses are antibody mediated. In an aspect, when tissue from the transgenic pig is transplanted into a human, an acute vascular rejection related symptom is decreased as compared to when tissue from a wild-type pig is transplanted into a human. In an aspect, when a liver from the transgenic pig is exposed to human platelets, the liver exhibits reduced uptake of human platelets as compared to when a liver from a wild-type pig is exposed to human platelets. In various embodiments, the nucleotide sequence is a human Class I HLA gene selected from the group of HLA MHC class I genes comprising HLA-A, HLA-A2, HLA-B, HLA-C, HLA-E, HLA-F, and HLA-G.

In an embodiment a skin related product obtained from a transgenic pig comprising a disrupted α(1,3)-galactosyltransferase (αGal) and SLA gene in the nuclear genome of at least one cell of the pig and wherein expression of αGal and SLA is decreased as compared to a wildtype pig and further comprising a nucleotide sequence encoding a Class I HLA polypeptide in the nuclear genome of at least one cell and wherein expression of HLA is increased as compared to a wild-type pig is provided. In an embodiment a skin related product obtained from a transgenic pig comprising a disrupted α(1,3)-galactosyltransferase, CMAH and SLA gene in the nuclear genome of at least one cell of the pig and wherein expression of α(1,3)-galactosyltransferase, CMAH and SLA is decreased as compared to a wild-type pig and further comprising a nucleotide sequence encoding a Class I HLA polypeptide in the nuclear genome of at least one cell and wherein expression of HLA is increased as compared to a wild-type pig is provided. In an embodiment, a skin related product obtained from a transgenic pig comprising a disrupted αGal, CMAH and SLA gene in the nuclear genome of at least one cell of the pig and wherein expression of αGal, CMAH and SLA is decreased as compared to a wildtype pig is provided. In an aspect of the application the skin related product exhibits reduced premature separation from a wound, particularly from a human skin wound.

Methods of preparing transplant material for xenotransplantation into a human are provided. The methods comprise providing a transgenic pig of the application as a source of the transplant material and wherein the transplant material is selected from the group consisting of organs, tissues, and cells and wherein the transplant material has reduced levels of SLA and αGal antigens and increased levels of HLA antigens, wherein the transplant material has reduced levels of αGal antigens, reduced levels of Neu5Gc antigens and reduced levels of SLA antigens and increased levels of HLA antigens or wherein the transplant material has reduced levels of of αGal antigens, reduced levels of Neu5Gc antigens and reduced levels of SLA antigens.

A transgenic pig comprising a disrupted α(1,3)-galactosyltransferase, CMAH and SLA gene and comprising a nucleotide sequence encoding an Class I HLA polypeptide in the nuclear genome of at least one cell of the pig is provided. Expression of α(1,3)-galactosyltransferase, CMAH and SLA in the transgenic pig is decreased as compared to expression in a wild-type pig and expression of the Class I HLA polypeptide is increased as compared to expression in a wild-type pig. A transgenic pig comprising a disrupted α(1,3)-galactosyltransferase, CMAH and SLA gene in the nuclear genome of at least one cell of the pig is provided. Expression of α(1,3)-galactosyltransferase, CMAH and SLA in the transgenic pig is decreased as compared to expression in a wild-type pig. A porcine organ, tissue or cell obtained from the transgenic pig is provided. A porcine organ, tissue or cell may be selected from the group consisting of skin, heart, liver, kidneys, lung, pancreas, thyroid, small bowel and components thereof. In an aspect, when tissue from the transgenic pig is transplanted into a human, a rejection related symptom is improved as compared to when tissue from a wild-type pig is transplanted into a human. Rejection related symptoms may occur as a result of cellular or humoral rejection responses. Such rejection responses may be acute or chronic. Cellular rejection responses are lymphocyte mediated; humoral rejection responses are antibody mediated. In an aspect, when tissue from the transgenic pig is transplanted into a human, an acute vascular rejection related symptom is decreased as compared to when tissue from a wild-type pig is transplanted into a human. In an aspect, when a liver from the transgenic pig is exposed to human platelets, the liver exhibits reduced uptake of human platelets as compared to when a liver from a wild-type pig is exposed to human platelets.

Transgenic pigs comprising disrupted α(1,3)-galactosyltransferase, CMAH and SLA genes and further comprising a nucleotide sequence encoding a Class I HLA polypeptide in the nuclear genome of at least one cell of the pig are provided. Transgenic pigs comprising disrupted α(1,3)-galactosyltransferase, CMAH and SLA genes in the nuclear genome of at least one cell of the pig are provided. In an embodiment, the disruption of the α(1,3)-galactosyltransferase gene is a three base pair deletion adjacent to a G to A substitution, a single base pair deletion, a six base pair deletion, a two base pair insertion, a ten base pair deletion, five base pair deletion, a seven base pair deletion, an eight base pair substitution for a five base pair deletion, a single base pair insertion, a five base pair insertion, and both a five base pair deletion and a seven base pair deletion, wherein the disruption of said CMAH gene is selected from the group of disruptions comprising twelve base pair deletion, a five base pair substitution for a three base pair deletion, a four base pair insertion, a two base pair deletion, an eight base pair deletion, a five base pair deletion, a three base pair deletion, a two base pair insertion for a single base pair deletion, a twenty base pair deletion, a one base pair deletion, an eleven base pair deletion, wherein the disruption of said SLA class I gene is selected from the group of disruptions comprising a 276 base pair deletion, a 276 base pair deletion in exon 4, a 4 base pair deletion, a 4 base pair deletion in exon 4, a 2 base deletion, a 1 base pair insertion, and a frameshift mutation in exon 4. In various embodiments the nucleotide sequence encoding the Class I HLA polypeptide is introduced into the SLA class I region. Expression of functional α(1,3)-galactosyltransferase, CMAH and SLA in the transgenic pig is decreased as compared to a wild-type pig; expression of a functional Class I HLA polypeptide in the transgenic pig is increased as compared to a wild-type pig. When tissue from the transgenic pig is transplanted into a human, a hyperacute rejection related syndrome is decreased as compared to when tissue from a wild-type pig is transplanted into a human.

Methods of increasing the duration of the period between when a human subject is identified as a subject in need of a human liver transplant and when said human liver transplant occurs are provided. The methods involve providing a liver from a transgenic pig comprising disrupted α(1,3)-galactosyltransferase, CMAH and SLA genes wherein expression of α(1,3)-galactosyltransferase, CMAH and a SLA product is decreased as compared to a wild-type pig and further comprising a nucleotide sequence encoding a functional Class I HLA polypeptide wherein expression of the HLA polypeptide is increased as compared to a wild-type pig and surgically attaching a liver from the transgenic pig to the human subject in a therapeutically effective manner. The methods involve providing a liver from a transgenic pig comprising disrupted α(1,3)-galactosyltransferase, CMAH and SLA genes wherein expression of α(1,3)-galactosyltransferase, CMAH and a SLA product is decreased as compared to a wild-type pig and surgically attaching a liver from the transgenic pig to the human subject in a therapeutically effective manner. In an aspect, the liver is surgically attached internal to the human subject. In an aspect, the liver is surgically attached external to the human subject. The liver may be directly or indirectly attached to the subject.

Methods of reducing premature separation of a skin related product from a human subject are provided. The methods involve the steps of providing a transgenic pig comprising disrupted α(1,3)-galactosyltransferase, CMAH and SLA genes and further comprising a nucleotide sequence encoding a Class I HLA polypeptide and preparing a skin related product from the transgenic pig. Expression of α(1,3)-galactosyltransferase, CMAH and SLA in the transgenic pig is decreased as compared to a wild-type pig; expression of a Class I HLA polypeptide in the transgenic pig is increased as compared to a wild-type pig. The methods involve the steps of providing a transgenic pig comprising disrupted α(1,3)-galactosyltransferase, CMAH and SLA genes and preparing a skin related product from the transgenic pig. Expression of α(1,3)-galactosyltransferase, CMAH and SLA in the transgenic pig is decreased as compared to a wild-type pig.

Methods of improving a hyperacute rejection related symptom in a patient are provided. The methods involve transplanting porcine transplant material having a reduced level of αGal antigens, a reduced level of SLA antigens and a reduced level of Neu5Gc antigens and an increased level of HLA antigens into a subject; the porcine transplant material may have HLA antigens rather than SLA antigens. Aspects of the methods involve transplanting porcine transplant material having a reduced level of αGal antigens, a reduced level of SLA antigens and a reduced level of Neu5Gc antigens into a subject. A hyperacute rejection related symptom is improved as compared to when porcine transplant material from a wild-type pig is transplanted into a human.

A cell culture reagent that exhibits an altered epitope profile is provided. The cell culture reagent is isolated from a transgenic pig comprising disrupted α(1,3)-galactosyltransferase, CMAH and SLA genes and further comprising a nucleotide sequence encoding a Class I HLA polypeptide. Expression of α(1,3)-galactosyltransferase, CMAH and SLA in the transgenic pig is decreased as compared to a wild-type pig; expression of a Class I HLA 1 polypeptide in the transgenic pig is increased as compared to a wild-type pig. In an aspect the cell culture reagent is isolated from a transgenic pig comprising disrupted α(1,3)-galactosyltransferase, CMAH and SLA genes. Expression of α(1,3)-galactosyltransferase, CMAH and SLA in the transgenic pig is decreased as compared to a wild-type pig. The cell culture reagent is selected from the group comprising cell culture media, cell culture serum, cell culture additives and isolated cells capable of proliferation. In an aspect, the cell culture reagent is isolated from a transgenic pig wherein the disruption of the α(1,3)-galactosyltransferase gene is a three base pair deletion adjacent to a G to A substitution, a single base pair deletion, a six base pair deletion, a two base pair insertion, a ten base pair deletion, five base pair deletion, a seven base pair deletion, an eight base pair substitution for a five base pair deletion, a single base pair insertion, a five base pair insertion, and both a five base pair deletion and a seven base pair deletion, wherein the disruption of said CMAH gene is selected from the group of disruptions comprising twelve base pair deletion, a five base pair substitution for a three base pair deletion, a four base pair insertion, a two base pair deletion, an eight base pair deletion, a five base pair deletion, a three base pair deletion, a two base pair insertion for a single base pair deletion, a twenty base pair deletion, a one base pair deletion, an eleven base pair deletion, wherein the disruption of said SLA class I gene is selected from the group of disruptions comprising a 276 base pair deletion, a 276 base pair deletion in exon 4, a 4 base pair deletion, a 4 base pair deletion in exon 4, a 2 base deletion, a 1 base pair insertion, and a frameshift mutation in exon 4, and wherein the nucleotide sequence encodes a Class I HLA polypeptides selected from the group of Class I HLA polypeptides including but not limited to HLA-A, HLA-B, HLA-C, HLA-E, HLA-F, HLA-G and HLA-A2.

Methods of producing a compound of interest with an altered epitope profile are provided. The method involves the steps of providing a cell culture reagent that exhibits an altered epitope profile and incubating an isolated cell capable of expressing the compound of interest with the cell culture reagent that exhibits an altered epitope profile. The cell culture reagent with an altered epitope profile is isolated from a transgenic pig comprising disrupted α(1,3)-galactosyltransferase, CMAH and SLA genes and further comprising a nucleotide sequence that encodes a Class I HLA polypeptide. Expression of α(1,3)-galactosyltransferase, CMAH and SLA in the transgenic pig is decreased as compared to a wild-type pig. The level of Neu5Gc, SLA or alphaGal epitopes on the compound of interest is lower than the level of Neu5Gc, SLA or alphaGal on the compound of interest when the compound of interest is produced from an isolated cell incubated with a cell culture reagent isolated from a wild-type pig and the level of Class I HLA epitopes on the compound of interest is higher than the level of HLA on the compound of interest when the compound of interest is produced from an isolated cell incubated with a cell culture reagent isolated from a wild-type pig. In an embodiment the compound of interest is selected from the group comprising glycolipids and glycoproteins. In various aspects, the compound of interest is a glycoprotein selected from the group of glycoproteins comprising antibodies, growth factors, cytokines, hormones and clotting factors. In an embodiment the disruption of the α(1,3)-galactosyltransferase gene is a three base pair deletion adjacent to a G to A substitution, a single base pair deletion, a six base pair deletion, a two base pair insertion, a ten base pair deletion, five base pair deletion, a seven base pair deletion, an eight base pair substitution for a five base pair deletion, a single base pair insertion, a five base pair insertion, and both a five base pair deletion and a seven base pair deletion, wherein the disruption of said CMAH gene is selected from the group of disruptions comprising twelve base pair deletion, a five base pair substitution for a three base pair deletion, a four base pair insertion, a two base pair deletion, an eight base pair deletion, a five base pair deletion, a three base pair deletion, a two base pair insertion for a single base pair deletion, a twenty base pair deletion, a one base pair deletion, an eleven base pair deletion, wherein the disruption of said SLA class I gene is selected from the group of disruptions comprising a 276 base pair deletion, a 276 base pair deletion in exon 4, a 4 base pair deletion, a 4 base pair deletion in exon 4, a 2 base deletion, a 1 base pair insertion, and a frameshift mutation in exon 4. wherein the nucleotide sequence encodes a Class I HLA polypeptides selected from the group of Class I HLA polypeptides including but not limited to HLA-A, HLA-B, HLA-C, HLA-E, HLA-F, HLA-G and HLA-A2.

Porcine transplant materials for transplantation into a human are provided. The porcine transplant material has a reduced level of αGal epitopes, a reduced level of at least one SLA epitope and a reduced level of Neu5Gc and an increased level of Class I HLA epitopes. In an aspect the porcine transplant material has a reduced level of αGal epitopes, a reduced level of at least one SLA epitope and a reduced level of Neu5Gc.

### BRIEF DESCRIPTION OF THE DRAWINGS

FIG. 1 provides information regarding swine SLA class I MHC genes. Panel A provides a schematic of the class I region of swine MHC. The class I region of swine MHC contains three classical class I genes (SLA-1, -2, -3;), several pseudogenes (SLA-4, -5, and -9) and two class I like genes (SLA-11 and -12). Panel B provides NCBI accession numbers that are relevant to the alleles of this study. Panel C depicts a cartoon of the five protein domains of the class I protein with an indication of which gene exon encodes each specific polypeptide region. The Δ₂m protein is also shown. Panel D provides a schematic showing the relative location of the gRNA targets in exon four of the class I gene. Panel E depicts the nucleotide sequences of several CRISPR gRNA in exon 4 of the class I target regions. SEQ ID NO:1, the target sequence of gRNA A, CCAGGACCAGAGCCAGGACATGG is shown in the top line of the chart. SEQ ID NO:2, the target sequence of gRNA B, GAGACCAGGCCCTCAGGGGATGG, is shown in the middle line of the chart. SEQ ID NO:3, the target sequence of gRNA C, CCAGAAGTGGGCGGCCCTGGTGG, is shown at the bottom of the chart.

FIG. 2 presents flow cytometry traces of fibroblast cells following gRNA-Cas9 Treatment and flow sorting. Following gRNA treatment, two successive rounds of flow cytometry sorting yielded class I negative SLA cells. A representative example of enrichment is shown (panel A). The isotype control peak in sort 2 is difficult to see because of overlap with the class I SLA histogram. When used singly or in combination, all three gRNA targeting exon four were capable of producing cells deficient in class I SLA expression (panel B).

FIG. 3 presents flow cytometry traces of cells from porcine fetuses. SCNT of fibroblasts isolated in FIG. 2 were used to create embryos. 32 days after impregnating a sow with these embryos, three fetuses were collected. Two of the fetuses were well formed and used to create fibroblast cultures. The fibroblasts were stained with a negative isotype control or with an antibody specific for class I SLA. Fetus-3 expressed low levels of SLA protein. Cells derived from Fetus-2 were devoid of class I SLA proteins.

FIG. 4 depicts results of phenotypic and cDNA Analyses of Class I SLA Deficient Piglets. In Panel A flow cytometry traces of fibroblasts from three piglets, recloned from the SLA negative fetal fibroblast cells isolated in FIG. 3, were examined for cell surface expression of class I SLA proteins. PBMC from piglets 2 and 3 were also evaluated. Traces obtained from cells isolated from the kidney (piglet-1) are also shown. Corresponding class I SLA positive cells are shown for comparison. Relative binding of class I specific SLA antibodies and an irrelevant isotype control are shown. Panel B is a photograph of amplified alleles of class I SLA separated by gel electrophoresis. cDNA, prepared from fetus-2 and piglets-1 and -2, were subjected to PCR with primers designed to amplify individual alleles of class I SLA. Sample W represents an identical analysis of the untreated parental, SLA expressing, fibroblasts. Samples F, 1, and 2 represent the fetus, and cloned animals 1 and 2 respectively.

FIG. 5 presents results of lymphocyte subset analysis of SLA expressing and SLA deficient pigs. PBMC were isolated from a class I SLA positive animal and two cloned pigs devoid of class I SLA molecules. Cells were incubated with a fluorescent viability dye, and antibodies specific for CD3, CD4, and CD8 molecules. Panel A provides a representative histogram shows the gating strategy to select for viable CD3 positive cells. Panel B shows CD4 and CD8 expression levels revealing each T cell subset. An isotype control staining was used to set the gates defining each subset. In Panel C, the means and standard deviations are shown for the various lymphocyte subsets (DN: CD4−CD8−, DP: CD4+CD8+, CD4: CD4+CD8−, CD8: CD4−CD8+) obtained from four separate PBMC isolations from the SLA positive animal and five separate PBMC isolations from the cloned animals (twice for Pig 2 and three times for Pig 3). Unpaired t tests were used to compare the frequencies of each cell type in SLA expressing and SLA deficient animals. P values are shown beneath the graph for comparison of the frequency of each subset between SLA positive and SLA negative animals.

FIG. 6 presents results of sequence analysis of SLA alleles in the SLA−/− transgenic fetus and two SLA− piglets (piglet-1 and piglet-2). The wild type sequence of the indicated allele is shown (WT).

FIG. 7 presents flow cytometry traces from SLA class II-deficient fetal fibroblasts. The top panels show flow cytometry traces obtained from untreated primary swine fetal fibroblasts. The bottom panels show flow cytometry traces obtained from primary swine fetal fibroblasts treated with gRNA and Cas9. The bottom right panel shows results from fetal fibroblasts treated with gRNA specific for SLA-DQ; the bottom left panel shows results from fetal fibroblasts treated with gRNA specific for SLA-DR. The area under the curve for the Class II SLA antibody is darkly shaded; the area under the curve for the isotype control is white; the area of overlap between the Class II SLA antibody and the isotype control is lightly shaded. The Class II antibody peak is clearly visible in the untreated cells and is not present for the cells treated with gRNA specific for swine class II SLA MHC and Cas9. Note the substantial peak shifts from both the SLA-DQ and SLD-DR cells in the lower panels as compared to the wildtype controls in the upper panels.

FIG. 8 presents data obtained from porcine kidneys obtained from GGTA1−/−/hDAF transgenic pigs and five rhesus macaques. Panel A shows anti-pig IgG antibody titers determined by flow cytometry analysis (xenograft crossmatch assay using GGTA1-/1 cells as targets) prior to transplant. Four of the five animals had low anti-pig IgG titers. Panel B presents creatinine levels (mg/dL) in the five rhesus macaques at the indicated time point past transplant (post-transplant days). Data from the macaque with a high titer of non-Gal antibody are shown. Data from the two animals with a low titer of antibody and treated with an anti-CD154 are shown. Data from the two animals with a low titer of antibody and treated with belatacept are shown. Creatinine levels were consistent in the anti-CD154 treated animals. Panel C presents platelet counts (Pits×1000) in the five rhesus macaques at the indicated time point past transplant (post-transplant days). Panel D presents images of analysis of a kidney from the high anti-pig IgG macaque which rejected the transplant less than one week post-transplant. The intact kidney is shown in the left image, and the dissected kidney is shown in the center left image. Micrographs of the histological examination are shown in the center right and right images. Graft interstitial hemorrhage and significant IgG and IgM deposition in the glomerular capillaries are present.

FIG. 9 panel A provides the primers used for the sgRNA exon 4 targets and panel B provides the primers used to amplify SLA DNA for various alleles.

FIG. 10 panel A presents flow cytometry traces of various cells stained with anti-SLA class I antibodies. Panel B presents flow cytometry traces of various cells stained with anti-B2M antibodies.

FIG. 11 presents graphs of interferon-γ Elispot assays performed with porcine aortic endothelial cells (AECs) from α-Gal pigs (SLA+ target, white bars) and with porcine aortic endothelial cells with an HLA-A2 gene in the class I SLA loci obtained from a knockout pig (HLA-A2+ target, solid bars) and human peripheral blood monocytes (PBMC) from either HLA-A2 positive (HLA-A2+, panel A) samples or HLA-A2 negative (HLA-A2−, panel B) samples. The porcine AEC functioned as the antigen to test xeno-antigen specific Interferon-γ (IFN) responses in the human PBMC samples. The number of IFNγ producing positive cells is shown on the y-axis. The PBMC sample indicator is shown on the x-axis.

FIG. 12 provides plots of data obtained from flow cytometry analysis of human antibody (IgG or IgM) binding to αGal- porcine AEC's expressing HLA-A2 and lacking SLA class I or expressing SLA class I. Flow cytometry analysis was performed as described elsewhere herein. The upper plots show IgG results; the lower plots show IgM results. The SLA+ results (x-axis) were plotted against the HLA-A2+/SLA− results (y-axis). Results obtained with HLA-A2 reactive serum are shown in the left plots. Results obtained with HLA-A2 non-reactive serum are shown in the right plots. Minimal changes are observed in the IgM results. The IgG results indicate greater IgG involvement in antibody binding.

FIG. 13 provides data obtained from HLA-A2 transfectants. The bar graph indicates relative expression of HLA-A2 in the presence (grey bar, IFN-gamma) or absence (empty bar, no treatment) of interferon-γ. HLA-A2 expression increases after treatment with IFN-γ, as expected for a sequence controlled by the IFN-γ responsive SLA-I promoter region. The histogram shows HLA−A2 expressing cells.

FIG. 14 provides a schematic of the Clal Trap2 PUC Hygro SLA-1 removal vector and HLA-SLA swap insertion. The entire removal vector construct is generally depicted as a series of regions (1-9). The SLA-1 homology arms are shown in regions 2 and 9. Upon transfection and successful insertion, the removal vector construct from region 2 through region 9 is inserted in the porcine genome (Correctly Inserted Construct in the Genome). After re-transfection with a recombinase, the selectable marker and the SV40 region (regions 4 and 5) are removed from the construct and the porcine genome. A diagram of the final insert without the selectable marker is shown (Post Recombination Construct).

### DETAILED DESCRIPTION OF THE INVENTION

The present application provides transgenic pigs and porcine organs, tissues and cells for transplantation into a human that do not express the indicated pig genome encoded products and methods of making and using the same. In one embodiment the application provides a triple transgenic pig comprising disrupted α(1,3)-galactosyltransferase, cytidine monophosphate-N-acetylneuraminic acid hydroxylase and SLA genes, wherein expression of functional α(1,3)-galactosyltransferase, cytidine monophosphate-N-acetylneuraminic acid hydroxylase and a SLA antigen in the transgenic pig is decreased as compared to a wild-type pig. In an embodiment the application provides a triple transgenic pig comprising disrupted α(1,3)-galactosyltransferase and SLA genes, wherein expression of functional α(1,3)-galactosyltransferase and a SLA antigen in the transgenic pig is decreased as compared to a wild-type pig and further comprising a nucleotide sequence encoding a Class I HLA polypeptide wherein expression of a Class I HLA polypeptide in increased as compared to a wildtype pig. In an embodiment the application provides a triple transgenic pig comprising disrupted α(1,3)-galactosyltransferase, cytidine monophosphate-N-acetylneuraminic acid hydroxylase and SLA genes, wherein expression of functional α(1,3)-galactosyltransferase, cytidine monophosphate-N-acetylneuraminic acid hydroxylase and a SLA antigen in the transgenic pig is decreased as compared to a wild-type pig and further comprising a nucleotide sequence encoding a Class I HLA polypeptide wherein expression of a Class I HLA polypeptide is increased as compared to a wild-type pig.

### I. In General

In the specification and in the claims, the terms “including” and “comprising” are open-ended terms and should be interpreted to mean “including, but not limited to . . . ” These terms encompass the more restrictive terms “consisting essentially of” and “consisting of”.

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. Compositions and Methods

Transgenic animals suitable for use in xenotransplantation and methods of producing mammals suitable for use in xenotransplantation are provided. Specifically, the present application describes the production of homozygous triple transgenic pigs with decreased expression of alpha 1,3 galactosyltransferase (αGal), cytidine monophosphate-N-acetylneuraminic acid hydroxylase (CMAH) and a swine leukocyte antigen (SLA). The present application describes homozygous transgenic pigs with increased expression of a Class I HLA polypeptide and decreased expression of alpha 1,3 galactosyltransferase (αGal), cytidine monophosphate-N-acetylneuraminic acid hydroxylase (CMAH) and a swine leukocyte antigen (SLA) or decreased expression of alpha 1,3 galactosyltransferase (αGal) and a swine leukocyte antigen (SLA).

In embodiments of the present invention, pigs and porcine organs, tissues and cells therefrom are provided in which the αGal, SLA and CMAH genes are less active, such that the resultant αGal, CMAH and SLA products no longer generate wild-type levels of α1,3-galactosyl epitopes, SLA epitopes or Neu5Gc on a cell surface, glycoprotein or glycolipid. In an alternative embodiment the αGal, SLA and CMAH genes are inactivated in such a way that no transcription of the gene occurs. Various embodiments encompass a triple alphaGal/SLA/CMAH transgenic product. Triple transgenic (GT/SLA/CMAH-KO) cells are encompassed by the embodiments. Methods of making transgenic pigs, and the challenges thereto, are discussed in Galli et al 2010 Xenotransplantation 17(6) p. 397-410. Methods and cell cultures of the invention are further detailed below herein.

The term “transgenic mammal” refers to a mammal wherein a given gene has been altered, removed or disrupted. It is to be emphasized that the term is to be intended to include all progeny generations. Thus, the founder animal and all F1, F2, F3 and so on progeny thereof are included, regardless of whether progeny were generated by somatic cell nuclear transfer (SCNT) from the founder animal or a progeny animal or by traditional reproductive methods. By “single transgenic” is meant a transgenic mammal wherein one gene has been altered, removed or disrupted. By “double transgenic” is meant a transgenic mammal wherein two genes have been altered, removed or disrupted. By “triple transgenic” is meant a transgenic mammal wherein three genes have been altered, removed or disrupted. By “quadruple transgenic” is meant a transgenic mammal wherein four genes have been altered, removed or disrupted.

In principle transgenic animals may have one or both copies of the gene sequence of interest disrupted. In the case where only one copy or allele of the nucleic acid sequence of interest is disrupted, the transgenic animal is termed a “heterozygous transgenic animal”. The term “null” mutation encompasses both instances in which the two copies of a nucleotide sequence of interest are disrupted differently but for which the disruptions overlap such that some genetic material has been removed from both alleles, and instances in which both alleles of the nucleotide sequence of interest share the same disruption. In various embodiments disruptions of the three genes of interest may occur in at least one cell of the transgenic animal, at least a plurality of the animal's cells, at least half the animal's cells, at least a majority of animal's cells, at least a supermajority of the animal's cells, at least 70%, 75″, 80%, 85%, 90%, 95%, 98%, or 99% of the animal's cells.

The term “chimera”, “mosaic” or “chimeric mammal” refers to a transgenic mammal with a transgenic in some of its genome-containing cells. A chimera has at least one cell with an unaltered gene sequence, at least several cells with an unaltered gene sequence or a plurality of cells with an unaltered sequence.

The term “heterozygote” or “heterozygotic mammal” refers to a transgenic mammal with a disruption on one of a chromosome pair in all of its genome containing cells.

The term “homozygote” or “homozygotic mammal” refers to a transgenic mammal with a disruption on both members of a chromosome pair in all of its genome containing cells. A “homozygous alteration” refers to an alteration on both members of a chromosome pair.

A “non-human mammal” of the application includes mammals such as rodents, sheep, dogs, ovine such as sheep, bovine such as beef cattle and milk cows, and swine such as pigs and hogs. Although the application provides a typical non-human animal (pigs), other animals can similarly be genetically modified.

A “mutation” is a detectable change in the genetic material in the animal that is transmitted to the animal's progeny. A mutation is usually a change in one or more deoxyribonucleotides, such as, for example adding, inserting, deleting, inverting or substituting nucleotides.

By “pig” is intended any pig known to the art including, but not limited to, a wild pig, domestic pig, mini pigs, a Sus scrofa pig, a Sus scrofa domesticus pig, as well as in-bred pigs. Without limitation the pig can be selected from the group comprising Landrace, Yorkshire, Hampshire, Duroc, Chinese Meishan, Chester White, Berkshire Goettingen, Landrace/York/Chester White, Yucatan, Bama Xiang Zhu, Wuzhishan, Xi Shuang Banna and Pietrain pigs. Porcine organs, tissues or cells are organs, tissues, devitalized animal tissues, or cells from a pig.

The alpha 1,3 galactosyltransferase (αGal, GGTA, GGT1, GT, αGT, GGTA1, GGTA-1) gene encodes an enzyme (GT, αGal, α1,3 galactosyltransferase). Ensemble transcript ENSSSCG00000005518 includes the porcine GGTA1 nucleotide sequence. Functional α1,3 galactosyltransferase catalyzes formation of galactose-α1,3-galactose (αGal, Gal, Gal, gal1,3gal, gal1-3gal) residues on glycoproteins. The galactose-α1,3-galactose (αGal) residue is an antigenic epitope or antigen recognized by the human immunological system. Removing αGal from transgenic organ material does not eliminate the human immunological response to transplant of foreign material, suggesting an involvement of additional antibodies in the rapid immunological response to xenotransplant. (Mohiudden et al (2014), Am J. Transplantation 14:488-489 and Mohiudden et al 2014 Xenotransplantation 21:35-45). Disruptions of the αGal gene that result in decreased expression of functional αGal may include but are not limited to a 3 base pair deletion adjacent to a G to A substitution, a single base pair deletion, a single base pair insertion, a two base pair insertion, a six base pair deletion, a ten base pair deletion, a seven base pair deletion, an eight base pair insertions for a five base pair deletion and a five base pair insertion (see Table 1). The Crispr target sequence is in exon 3 of the gene, near the start codon.

Swine produce swine leukocyte antigens (SLA) from multiple SLA genes. Humans and non-human primate CD8+ and CD4+ T cells can be activated by SLA Class I and II, respectively. SLA's are characterized in a class selected from the group comprising Class I and Class II. SLA genes include, but are not limited to SLA-1, SLA-2, SLA-3, SLA-4, SLA-5, SLA-9, SLA-11, SLA-DQ and SLA-DR. SLA-1, SLA-2 and SLA-3 are SLA Class I (SLA1) genes. SLA-DQ and SLA-DR are SLA Class II genes. Anti-SLA class 1 (anti-SLA1) antibodies may react with products of the SLA-1, SLA-2 and SLA-3 genes. The SLA-1*0702 allele sequence is available as Genbank Acc. No: EU440330.1. The SLA-1*1201 allele sequence is available as Genbank Acc. No: EU440335.1. The SLA-1*1301 allele sequence is available as Genbank Acc. No: EU440336.1. The SLA-2 1001 allele sequence is available as Genbank Acc. No: EU432084.1. The SLA-2 2002 allele sequence is available as Genbank Acc. No: EU432081.1. The SLA-3*0402 allele sequence is available as Genbank Acc. No: EU432092.1. The SLA-3*0502 allele sequence is available as Genbank Acc. No: EU432094.1. Transgenic pigs expressing a dominant negative version of the human class I transactivator (CIITA), a transcription factor critical for expression of SLA class II have been created. The CIITA expressing pigs appeared healthy and viable. In the CIITA pigs, class II SLA expression was reduced by 40-50%. See Hara et al 2013, “Human dominant-negative class II transactivator transgenic pigs-effect on the human anti-pig T-Cell immune response and immune status”, Immunol 140:39-46, herein incorporated by reference in their entirety.

Human Leukocyte Antigens (HLA) molecules are the cell surface receptors of the major histocompatibility complex (MHC) in humans. Class I and Class II MHC's are significantly involved in transplant recognition and rejection. Matching HLA genes between donors and recipients reduces transplant rejection. An embodiment of the application provides HLA on porcine cells reduce transplant rejection.

The cytidine monophosphate-N-acetylneuraminic acid hydroxylase (CMP-Neu5Ac hydroxylase gene, CMAH) gene encodes an enzyme (CMAH). Functional CMAH catalyzes conversion of sialic acid N-acetylneuraminic acid (Neu5Ac) to N-glycolylneuraminic acid (Neu5Gc). The Neu5Gc residue is an antigenic epitope or antigen recognized by the human immunological system. The Ensembl database id Gene: ENSSSCG00000001099 includes the porcine CMAH nucleotide sequence. The Crispr target area is near exon 6. Disruptions of the CMAH gene that result in decreased expression of functional CMAH may include but are not limited to a four base pair insertion, a one base pair deletion, a two base pair deletion, a three base pair deletion, a five base pair deletion, an eight base pair deletion, an eleven base pair deletion, a twelve base pair deletion, a single base pair insertion, a two base pair insertion for single base pair deletion, and a three base pair deletion for a five base pair insertion

TABLE 1
Examples of Disruptions of Genes of Interest in Viable Pigs with Decreased
Functional Gene Product

| | | Disruption |
|---|---|---|
| | | Class |
| wildtype | disruption | Descriptor |
| GGTA1-GTCATCTTTTACATCATGGTGGAT | GGTA1- | 3 base pair |
| GATATCTCCAGGATGCC | GTCATCTTTTACATCATG___ | deletion |
| | AAT GATATCTCCAGGATGCC | adjacent to |
| | | a G to A |
| | | substitution |
| GGTA1- | GGTA1- | Single base |
| CTTTTCCCAG GAGAAAATAAT GAATGTCAAA | CTTTTCCCAG GAGAAAATAAT | pair |
| GGAAGAGTGG TTCT | GAATGT_AAA GGAAGAGTGG | deletion |
| | TTCT | |
| GGTA1- | GGTA1- | 6 base pair |
| CTTTTCCCAG GAGAAAATAAT GAATGTCAAA | CTTTTCCCAG GAGAAAATAAT | deletion |
| GGAAGAGTGG TTCT | _______CAAA | |
| | GGAAGAGTGG TTCT | |
| GGTA1- | GGTA1- | 2 base pair |
| CTTTTCCCAG GAGAAAATAAT GAATGTCAAA | CTTTTCCCAG GAGAAAATAAT | insertion |
| GGAAGAGTGG TTCT | GAATGTATCAAA | |
| | GGAAGAGTGG TTCT | |
| GGTA1- | GGTA1- | 10 base |
| CTTTTCCCAG GAGAAAATAAT GAATGTCAAA | CTTTTCCCAG GAGAAAATAA_ | pair |
| GGAAGAGTGG TTCT | _________A | deletion |
| | GGAAGAGTGG TTCT | |
| GGTA1- | GGTA1 | 7 base pair |
| CTTTTCCCAG GAGAAAATAAT GAATGTCAAA | CTTTTCCCAG GAGAAAATAAT | deletion |
| GGAAGAGTGG TTCT | GAA____ ___ | |
| | GGAAGAGTGG TTCT | |
| GGTA1- | GGTA1 | 7 base pair |
| GAGAAAATAAT GAATGTCAAAGG | GAGAAAATAAT G____ ___ | deletion |
| | AAGG | |
| GGTA1- | GGTA1 | 8 base pair |
| CTTTTCCCAG GAGAAAATAAT GAATGTCAAA | CTTTTCCCAG GAGAAAATAAT | substitution |
| GGAAGAGTGG TTCT | GAA GGAATAAT AA | for 5 base |
| | GGAAGAGTGG TTCT | pair |
| | | deletion |
| GGTA1- | GGTA1- | Single base |
| CTTTTCCCAG GAGAAAATAAT GAATGTCAAA | CTTTTCCCAG GAGAAAATAAT | pair |
| GGAAGAGTGG TTCT | GAATGTTCAAA | insertion |
| | GGAAGAGTGG TTCT | |
| GGTA1- | GGTA1- | 5 base pair |
| GAGAAAATAAT GAATGTCAAAGG | GAGAAAATAAT_____ | insertion |
| | TCAAAGG | |
| CMAH- | CMAH- | 4 base pair |
| AAACTCCTGA ACTACAAGGC TCGGCTGGTG | AAACTCCTGA | insertion |
| AAGGA | ACTACAAGGAA GGC | |
| | TCGGCTGGTG AAGGA | |
| CMAH- | CMAH- | 2 base pair |
| CAGGCGTGAG TAAGGTACGT GATCTGTTGGA | CAGGCGTGAG TAAGGTACGT | deletion |
| AGACAGTGA GATTCAGATGAT | GATC__TTGGA AGACAGTGA | |
| | GATTCAGATGAT | |
| CMAH- | CAGGCGTGAG TAAGGTACGT | 8 base pair |
| CAGGCGTGAG TAAGGTACGT GATCTGTTGGA | G________GA | deletion |
| AGACAGTGA GATTCAGATGAT | AGACAGTGA GATTCAGATGAT | |
| CMAH- | CAGGCGTGAG TAAGGTACGT | 5 base pair |
| CAGGCGTGAG TAAGGTACGT GATCTGTTGGA | G_____ TTGGA | deletion |
| AGACAGTGA GATTCAGATGAT | AGACAGTGA GATTCAGATGAT | |
| CMAH- | CAGGCGTGAG TAAGGTACGT | 3 base pair |
| CAGGCGTGAG TAAGGTACGT GATCTGTTGGA | GA___ GTTGGA | deletion |
| AGACAGTGA GATTCAGATGAT | AGACAGTGA GATTCAGATGAT | |
| CMAH- | CAGGCGTGAG TAAGGTACGT | 2 base pair |
| CAGGCGTGAG TAAGGTACGT GATCTGTTGGA | GATCACGTTGGA | insertion for |
| AGACAGTGA GATTCAGATGAT | AGACAGTGA GATTCAGATGAT | single base |
| | | pair |
| | | deletion |
| CMAH- | CAGGCGTGAG TAAGGTACGT | 20 base |
| CAGGCGTGAG TAAGGTACGT GATCTGTTGGA | GAT_______________ | pair |
| AGACAGTGA GATTCAGATGAT | _____TCAGATGAT | deletion |
| CMAH- | CAGGCGTGAG TAAGGTACGT | 1 base pair |
| CAGGCGTGAG TAAGGTACGT GATCTGTTGGA | GATCTTGTTGGA | insertion |
| AGACAGTGA GATTCAGATGAT | AGACAGTGA GATTCAGATGAT | |
| CMAH- | CAGGCGTGAG TAAGGTACGT | 1 base pair |
| CAGGCGT GAG TAAGGTACGT GATCTGTTGGA | GATC_GTTGGA AGACAGTGA | deletion |
| AGACAGTGA GATTCAGATGAT | GATTCAGATGAT | |
| CMAH | CMAH | 11 base |
| GAGTAAGGTACG TGATCTGTTGG AAGACAGT | GAGTAAGG__________ | pair |
| | _ TTGG AAGACAGT | deletion |
| CMAH | CMAH | 12 base |
| GAGTAAGGTACG TGATCTGTTGG AAGACAGT | GAGTAAGGTACG TGA____ | pair |
| | ________CAGT | deletions |
| CMAH | CMAH | 3 base pair |
| GAGTAAGGTACG TGATCTGTTGGAAGACAGT | GAGTAAGGTACG TGAGTAAG | deletion 5 |
| | TTGG AAGACAGT | base pair |
| | | insertion |
| SLA-1*0702 | SLA-1 | 276 base |
| GCCTCCAAAGACACATGTGACCCGCCACCCCAGC | | pair |
| TCTGACCTGGGGGTCACCTTGAGGTGCTGGGCCC | | deletion in |
| TGGGCTTCTACCCTAAGGAGATCTCCCT | | exon 4 |
| GACCTGGCAGCGGGAGGGCCAGGACCAGAGCCA | | |
| GGACATGGAGCTGGTGGAGACCAGGCCCTCAGG | | |
| GGATGGGACCTTCCAGAAGTGGGCGGCCC | | |
| TGGTGGTGCCTCCTGGAGAGGAGCAGAGCTACAC | | |
| CTGCCATGTGCAGCACGAGGGCCTGCAGGAGCCC | | |
| CTCACCCTGAGATGGGA | | |
| SLA-1*0702 | SLA-1 | 4 base pair |
| GGCCCAGGACCAGAGCCAGGACATGGAGCTGGT | GGCCCAGGACCAGAGCCA__ | deletion |
| GG | __ATGGAGCTGGTGG | |
| SLA-1*1301/*1001 recombinant | GGCCCAGGACCAGAGCC__ | 2 base pair |
| GGCCCAGGACCAGAGCCAGGACATGGAGCTGGT | GACATGGAGCTGGTGG | deletion |
| GG | | |
| SLA-1*1001/SLA-12 recombinant | GGCCCAGGACCAGAGCCAGG | 1 base pair |
| GGCCCAGGACCAGAGCCAGGACATGGAGCTGG | gACATGGAGCTGG | insertion |

Transgenic Animals. The present invention provides a transgenic animal lacking any expression of functional αGal and CMAH genes and reduced expression of one or more SLA genes. The animal can be any mammal suitable for xenotransplantation. In a specific embodiment, the animal is a pig. “CMAH/αGAL double knockout”, “CMAH/αGAL DKO”, “CMAH/αGal”, “CMAH/αGal DKO”, “CMAH^{-/-}/GAL^{-/-}”, “αGal/CMAH DKOs”, “αGAL/CMAH double knockouts”, “GGTA1/CMAH DKO”, “GT1/CMAH DKO”, “GGTA1^{-/-}/CMAH^{-/-}”, “GGT1^{-/-}/CMAH^{-/-}”, “CMAH/GGTA DKO”, “GT/CMAH-KO”, “GGTA1/CMAH KO”, “DKO (αGal/CMAH)”, “DKO (αGAL & CMAH)”, “CMAH-/αGal-”, “αGal-/CMAH-”, “CMAH-/αGAL-” and variants thereof refer to animals, cells, or tissues that lack expression of functional alpha 1,3 galactosyltransferase and cytidine monophosphate-N-acetylneuraminic acid hydroxylase. A triple transgenic product or pig may be created in a wild-type background or in a CMAH/αGal double knockout background.

The phrase “disrupted gene” is intended to encompass insertion, interruption, or deletion of a nucleotide sequence of interest wherein the disrupted gene either encodes a polypeptide having an altered amino acid sequence that differs from the amino acid sequence of the endogenous sequence, encodes a polypeptide having fewer amino acid residues than the endogenous amino acid sequence or does not encode a polypeptide although the nucleotide sequence of interest encodes a polypeptide.

The present specification provides a transgenic animal with reduced expression of functional αGal, SLA and CMAH genes. In one embodiment the transgenic animal lacks expression of functional αGal, CMAH and a class I SLA. In another embodiment the transgenic animal lacks expression of functional αGal, CMAH and a class II SLA. In another embodiment the transgenic animal lacks expression of functional αGal, CMAH, a class I SLA and a class II SLA. In yet another embodiment the transgenic animal lacks expression of functional αGal, CMAH and more than one SLA Class I genes or more than one SLA Class II genes. In still another embodiment, the transgenic animal lacks expression of functional αGal, CMAH, more than one SLA Class 1 gene and at least one SLA Class II gene. In yet still another embodiment, the transgenic animal lacks expression of functional αGal, CMAH, more than one SLA Class II gene and at least on SLA Class 1 gene. In another embodiment the transgenic animal lacks expression of functional αGal and at least one SLA Class I gene. In another embodiment the transgenic animal lacks expression of functional αGal and at least one SLA Class II gene. The animal can be any mammal suitable for xenotransplantation. In a specific embodiment, the animal is a pig. In an embodiment the transgenic animal has reduced expression of functional αGal, SLA, B4GaINT2 and CMAH genes. αGal, SLA and CMAH transgenic pigs may be further altered to express inhibitory or co-inhibitory molecules or by removing additional molecules including but not limited to ASGR1, vWF, Mac-1 (CR3, complement receptor 3), CD11 b or CD18.

The present invention provides a transgenic animal with increased expression of a Class I HLA polypeptide and reduced expression of functional αGal, SLA and CMAH genes. In one embodiment the transgenic animal lacks expression of functional αGal, CMAH and a class I SLA. In another embodiment the transgenic animal lacks expression of functional αGal, CMAH and a class II SLA. In another embodiment the transgenic animal lacks expression of functional αGal, CMAH, a class I SLA and a class II SLA. In yet another embodiment the transgenic animal lacks expression of functional αGal, CMAH and more than one SLA Class I genes or more than one SLA Class II genes. In still another embodiment, the transgenic animal lacks expression of functional αGal, CMAH, more than one SLA Class 1 gene and at least one SLA Class II gene. In yet still another embodiment, the transgenic animal lacks expression of functional αGal, CMAH, more than one SLA Class II gene and at least on SLA Class 1 gene. In another embodiment the transgenic animal has increased expression of a Class I HLA polypeptide and lacks expression of functional αGal and at least one SLA Class I gene.

Transgenic transplant material. Transplant material encompasses organs, tissue and/or cells from an animal for use as xenografts. Transplant material for use as xenografts may be isolated from transgenic animals with decreased expression of αGal, SLA and CMAH. Transgenic transplant material from transgenic pigs can be isolated from a prenatal, neonatal, immature or fully mature animal. The transplant material may be used as temporary or permanent organ replacement for a human subject in need of an organ transplant. Any porcine organ can be used including, but not limited to, the brain, heart, lung, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, small bowel, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes and lymph vessels.

In another embodiment, the application provides non-human tissues that are useful for xenotransplantation. In various embodiments, the non-human tissue is porcine tissue from a triple αGal/CMAH/SLA transgenic pig. Any porcine tissue can be used including but not limited to, epithelium, connective tissue, blood, bone, cartilage, muscle, nerve, adenoid, adipose, areolar, brown adipose, cancellous muscle, cartilaginous, cavernous, chondroid, chromaffin, dartoic, elastic, epithelial, fatty, fibrohyaline, fibrous, Gamgee, gelatinous, granulation, gut-associated lymphoid, skeletal muscle, Haller's vascular, indifferent, interstitial, investing, islet, lymphatic, lymphoid, mesenchymal, mesonephric, multilocular adipose, mucous connective, myeloid, nasion soft, nephrogenic, nodal, osteoid, osseus, osteogenic, retiform, periapical, reticular, smooth muscle, hard hemopoietic and subcutaneous tissue, devitalized animal tissues including heart valves, skin, and tendons, and vital porcine skin.

Another embodiment provides cells and cell lines from porcine triple transgenic animals with reduced or decreased expression of αGal, SLA and CMAH. In one embodiment these cells or cell lines can be used for xenotransplantation. Cells from any porcine tissue or organ can be used including, but not limited to: epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, granulosa cells, cumulus cells, epidermal cells, endothelial cells, Islet of Langerhans cells, pancreatic insulin secreting cells, pancreatic alpha-2 cells, pancreatic beta cells, pancreatic alpha-1 cells, bone cells, bone precursor cells, neuronal stem cells, primordial stem cells, hepatocytes, aortic endothelial cells, microvascular endothelial cells, umbilical vein endothelial cells, fibroblasts, liver stellate cells, aortic smooth muscle cells, cardiac myocytes, neurons, Kupffer cells, smooth muscle cells, Schwann cells, erythrocytes, platelets, neutrophils, lymphocytes, monocytes, eosinophils, basophils, adipocytes, chondrocytes, pancreatic islet cells, thyroid cells, parathyroid cells, parotid cells, glial cells, astrocytes, red blood cells, white blood cells, macrophages, somatic cells, pituitary cells, adrenal cells, hair cells, bladder cells, kidney cells, retinal cells, rod cells, cone cells, heart cells, liver cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, dopaminergic cells, squamous epithelial cells, osteocytes, osteoblasts, osteoclasts, embryonic stem cells, fibroblasts and fetal fibroblasts.

In an embodiment the application provides non-human material suitable for transfusions from multiple transgenic porcine animals with reduced expression of αGal and a SLA gene. These materials suitable for transfusions may include, but are not limited to, blood, whole blood, plasma, serum, red blood cells, platelets, and white bloods cells. Such materials may be isolated, enriched or purified. Methods of isolating, enriching or purifying material suitable for transfusion are known in the art.

Nonviable derivatives include tissues stripped of viable cells by enzymatic or chemical treatment these tissue derivatives can be further processed through crosslinking or other chemical treatments prior to use in transplantation. In a preferred embodiment, the derivatives include extracellular matrix derived from a variety of tissues, including skin, bone, urinary, bladder or organ submucosal tissues. In addition, tendons, joints, and bones stripped of viable tissue to including but not limited to heart valves and other nonviable tissues as medical devices are provided. In an embodiment, serum or medium suitable for cell culture and isolated from a transgenic pig of the invention are provided. Components of porcine transgenic organs, tissues or cells are also provided. Components may also be modified through any means known in the art including but not limited to crosslinking and aldehyde crosslinking. Components may vary depending on the larger organ or tissue from which the component is obtained. Skin components may include but are not limited to stripped skin, collagen, epithelial cells, fibroblasts and dermis. Bone components may include but are not limited to collagen and extracellular matrix. Heart components may include but are not limited to valves and valve tissue.

“Xenotransplantation” encompasses any procedure that involves the transplantation, implantation or infusion of cells, tissues or organs into a recipient subject from a different species. Xenotransplantation in which the recipient is a human is particularly envisioned. Thus xenotransplantation includes but is not limited to vascularized xenotransplant, partially vascularized xenotransplant, unvascularized xenotransplant, xenodressings, xenobandages, xenotransfusions, and xenostructures.

In embodiments, cell culture reagents isolated from a transgenic pig comprising disrupted α(1,3)-galactosyltransferase, SLA and CMAH genes are provided. Cell culture reagents are reagents utilized for tissue culture, in vitro tissue culture, microfluidic tissue culture, cell culture or other means of growing isolated cells or cell lines. Cell culture reagents may include but are not limited to cell culture media, cell culture serum, a cell culture additive, a feeder cell, and an isolated cell capable of proliferation. By an “isolated cell capable of proliferation” is intended a cell isolated or partially isolated from other cell types or other cells wherein the cell is capable of proliferating, dividing or multiplying into at least one additional clonal cell.

Cells grown in culture may synthesize or metabolically incorporate antigenic epitopes into a compound of interest produced by the cultured cell. The antigenic epitopes may result in increased binding by human antibodies and decreased efficacy of the compound of interest. See Ghaderi et al 2010 Nature Biotechnology 28(8):863-867, herein incorporated by reference in its entirety. Growing the producing cell in a cell culture reagent with an altered epitope profile such as a reduced level of αGal, SLA or Neu5Gc may reduce the level of αGal antigens, SLA antigens, or Neu5Gc antigens, or αGal, SLA Neu5Gc antigens combined on the compound of interest. Compounds of interest may include but are not limited to glycoproteins and glycolipids. Glycoproteins of interest may include but are not limited to an antibody, growth factor, cytokine, hormone or clotting factor. Glycolipids of interest may include but are not limited to therapeutics, antigens, and bio-surfactants.

The word “providing” is intended to encompass preparing, procuring, getting ready, making ready, supplying or furnishing. It is recognized that methods of providing a cell may differ from methods of providing a subject, methods of providing an organ may differ from methods of providing a pig, methods of providing a kidney may differ from methods of providing a liver and methods of providing an organ may differ from methods of providing a material suitable for transfusion.

Transplant rejection occurs when transplanted tissue, organs, cells or material are not accepted by the recipients body. In transplant rejection, the recipient's immune system attacks the transplanted material. Multiple types of transplant rejection exist and may occur separately or together. Rejection processes included but are not limited to hyperacute rejection (HAR), acute humoral xenograft rejection reaction (AHXR), thrombocytopenia, acute humoral rejection, hyperacute vascular rejection, antibody mediated rejection and graft versus host disease. By “hyperacute rejection” we mean rejection of the transplanted material or tissue occurring or beginning within the first 24 hours post-transplant involving one or more mechanisms of rejection. Rejection encompasses but is not limited to “hyperacute rejection”, “humoral rejection”, “acute humoral rejection”, “cellular rejection” and “antibody mediated rejection”. The acute humoral xenograft reaction (AHXR) is characterized by a spectrum of pathologies including, but not limited to, acute antibody mediated rejection occurring within days of transplant, the development of thrombotic microangiopathy (TMA), microvascular angiopathy, pre-formed non-Gal IgM and IgG binding, complement activation, microvascular thrombosis and consumptive thrombocytopenia within the first few weeks post transplant. Thrombocytopenia is a quantity of platelets below the normal range of 140,000 to 440,000/μl. Thrombocytopenia related symptoms include, but are not limited to, internal hemorrhage, intracranial bleeding, hematuria, hematemesis, bleeding gums, abdominal distension, melena, prolonged menstruation, epistaxis, ecchymosis, petechiae or purpura. Uptake of human platelets by pig livers contributes to the development of thrombocytopenia in xenograft recipients. Thrombocytopenia may occur upon reperfusion of the xenotransplanted organ or after the immediate post-reperfusion period.

In another embodiment, the invention provides a method of improving a rejection related symptom in a patient comprising transplanting porcine organs, tissue or cells having reduced expression of αGal, SLA and Neu5Gc on the porcine organs, tissue or cells into a human, wherein one or more rejection related symptoms is improved as compared to when tissue from a wild-type swine is transplanted into a human. By “improving”, “bettering”, “ameliorating”, “enhancing”, and “helping” is intended advancing or making progress in what is desirable. It is also envisioned that improving a rejection related symptom may encompass a decrease, lessening, or diminishing of an undesirable symptom. It is further recognized that a rejection related symptom may be improved while another rejection related symptom is altered. The altered second rejection related symptom may be improved or increased. A second altered rejection related symptom may be altered in a less desirable manner. Rejection related symptoms include but are not limited to hyperacute rejection related symptoms and acute humoral xenograft reaction related symptoms. Rejection related symptoms may include, but are not limited to, thrombotic microangiopathy (TMA), microvascular angiopathy, pre-formed non-Gal IgM and IgG binding, complement activation, agglutination, fibrosis, microvascular thrombosis, consumptive thrombocytopenia, consumptive coagulopathy, profound thrombocytopenia, refractory coagulopathy, graft interstitial hemorrhage, mottling, cyanosis, edema, thrombosis, necrosis, fibrin thrombi formation, systemic disseminated intravascular coagulation, IgM deposition in glomerular capillaries, IgG deposition in glomerular capillaries, elevated creatinine levels, elevated BUN levels, T cell infiltrate, infiltrating eosinophils, infiltrating plasma cells, infiltrating neutrophils, arteritis, antibody binding to endothelium, altered expression of ICOS, CTLA-4, BTLA, PD-1, LAG-3, or TIM-3, and systemic inflammation.

“Hyperacute rejection related symptom” is intended to encompass any symptom known to the field as related to or caused by hyperacute rejection. It is recognized that hyperacute rejection related symptoms may vary depending upon the type of organ, tissue or cell that was transplanted. Hyperacute rejection related symptoms may include, but are not limited to, thrombotic occlusion, hemorrhage of the graft vasculature, neutrophil influx, ischemia, mottling, cyanosis, edema, organ failure, reduced organ function, necrosis, glomerular capillary thrombosis, lack of function, hemolysis, fever, clotting, decreased bile production, asthenia, hypotension, oliguria, coagulopathy, elevated serum aminotransferase levels, elevated alkaline phosphatase levels, jaundice, lethargy, acidosis and hyperbilirubenemia and thrombocytopenia.

Any method of evaluating, assessing, analyzing, measuring, quantifying, or determining a rejection related symptom known in the art may be used with the claimed compositions and methods. Methods of analyzing a rejection related symptom may include, but are not limited to, laboratory assessments including CBC with platelet count, coagulation studies, liver function tests, flow cytometry, immunohistochemistry, standard diagnostic criteria, immunological methods, western blots, immunoblotting, microscopy, confocal microscopy, transmission electron microscopy, IgG binding assays, IgM binding assays, expression asays, creatinine assays and phagosome isolation.

Expression of a gene product is decreased when total expression of the gene product is decreased, a gene product of an altered size is produced or when the gene product exhibits an altered functionality. Thus if a gene expresses a wild-type amount of product but the product has an altered enzymatic activity, altered size, altered cellular localization pattern, altered receptor-ligand binding or other altered activity, expression of that gene product is considered decreased. Expression may be analyzed by any means known in the art including, but not limited to, RT-PCR, Western blots, Northern blots, microarray analysis, immunoprecipitation, radiological assays, polypeptide purification, spectrophotometric analysis, Coomassie staining of acrylamide gels, ELISAs, 2-D gel electrophoresis, in situ hybridization, chemiluminescence, silver staining, enzymatic assays, ponceau S staining, multiplex RT-PCR, immunohistochemical assays, radioimmunoassay, colorimetric assays, immunoradiometric assays, positron emission tomography, fluorometric assays, fluorescence activated cell sorter staining of permeablized cells, radioimunnosorbent assays, real-time PCR, hybridization assays, sandwich immunoassays, flow cytometry, SAGE, differential amplification or electronic analysis. Expression may be analyzed directly or indirectly. Indirect expression analysis may include but is not limited to, analyzing levels of a product catalyzed by an enzyme to evaluate expression of the enzyme. See for example, Ausubel et al, eds (2013) Current Protocols in Molecular Biology, Wiley-Interscience, New York, N.Y. and Coligan et al (2013) Current Protocols in Protein Science, Wiley-Interscience New York, N.Y.

“As compared to” is intended to encompass comparing something to a similar but separate thing, such as comparing a data point obtained from an experiment with a transgenic pig to a data point obtained from a similar experiment with a wildtype pig. The word “comparing” is intended to encompass examining character, qualities, values, quantities, or ratios in order to discover resemblances or differences between that which is being compared. Comparing may reveal a significant difference in that which is being compared. By “significant difference” is intended a statistically significant difference in results obtained for multiple groups such as the results for material from a transgenic pig and material from a wild-type pig or results for material from a triple transgenic product or pig and material from a double transgenic product or pig. Generally statistical significance is assessed by a statistical significance test such as but not limited to the student's t-test, Chi-square, one-tailed t-test, two-tailed t-test, ANOVA, Dunett's post hoc test, Fisher's test and z-test. A significant difference between two results may be results with a p<0.1, p<0.05, p<0.04, p<0.03, p<0.02, p<0.01 or greater.

The word “isolated” is intended to encompass an entity that is physically separated from another entity or group. An isolated cell is physically separated from another group of cells. Examples of a group of cells include, but are not limited to, a developing cell mass, a cell culture, a cell line, a tissue, an organ and an animal. The word “isolating” is intended to encompass physically separating an entity from another entity or group. Examples include physically separating a cell from other cells, physically separating a cell component from the remainder of the cell and physically separating tissue or organ from an animal. An isolated cell or cell component is separated by 10%, 15%, 20%, 25%, 30%, 35%, 40%, 45%, 50%, 55%, 60%, 65%, 70%, 75%, 80%, 85%, 90%, 95%, 96%, 97%, 98%, 99%, up to 100% of other naturally occurring cells or cell components. Methods for isolating one or more cells from another group of cells are known in the art. See for example Freshney (ED) Culture of Animal Cells: a manual of basic techniques (3^{rd} Ed.) 1994, Wiley-Liss; Spector et al (Eds) (1998) Cells: a Laboratory Manual (vol. 1) Cold Spring Harbor Laboratory Press and Darling et al (1994) Animal Cells: culture and media John Wiley & Sons. Methods of isolating a tissue or an organ from an animal are known in the art and vary depending on the tissue or organ to be isolated and the desired method of transplanting the tissue or organ. Methods of isolating a transfusion product from an animal or sample are known in the art and vary depending on the desired transfusion product. Such methods include but are not limited to centrifugation, dialysis, elution, apheresis and cryoprecipitation.

A “skin related product” encompasses products isolated from skin and products intended for use with skin. Skin related products isolated from skin or other tissues may be modified before use with skin. Skin related products include but are not limited to replacement dressings, burn coverings, dermal products, replacement dermis, dermal fibroblasts, collagen, chondroitin, connective tissue, keratinocytes, cell-free xenodermis, cell-free pig dermis, composite skin substitutes and epidermis and temporary wound coverings. See for example Matou-Kovd et al (1994) Ann Med Burn Club 7:143, herein incorporated by reference in its entirety.

The attachment period of a skin related product is the time between application of the skin related product to a human subject and natural separation of the skin related product from the human subject. When a human subject's skin wound has sealed, a skin related product may be removed by natural separation or mechanical separation. However natural separation of a skin related product from a human subject may occur prematurely. Premature natural separation occurs before separation is desired by a medical practitioner. By way of example and not limitation, premature natural separation may occur before the wound has been sealed. Premature natural separation may also be termed “sloughing”, “shedding”, or “flaking”. Clinical management of premature natural separation may include reapplication of a skin related product, dressing application, bandage application, administering antibiotic, and administering fluids. A skin wound may be sealed by any means known in the art including but not limited to by growth of the subject's skin and by skin grafting. Reduced premature separation encompasses a decreased, lower, less frequent, diminished, smaller amount of natural separation of a skin related product before separation is desired by a medical practitioner. The reduced premature separation may relate to a lower number of complete, a lower number of partial premature separation events, and involvement of a smaller portion of the skin related product in a partial premature separation event than compared to a skin related product obtained from a wild-type pig. A skin related product of the instant application may also exhibit an increased, lengthened, improved, extended, or expanded attachment period. Use of a skin related product of the instant application may increase the duration of the attachment period.

A skin wound encompasses any injury to the integument including but not limited to an open wound, burn, laceration, ulcer, leg ulcer, foot ulcer, melanoma removal, cancer removal, plastic surgery, and bite.

By “surgically attaching” is intended joining, combining, uniting, attaching, fastening, connecting, joining or associating through any surgical method known in the art.

The efficiency of producing genetically modified pigs increases when SCNT is performed primarily with genetically modified cells. The process of making genetic modifications in pig cells is less than 100% efficient. Phenotypic sorting of targeted cells simplifies the process of isolating modified cells from the whole population of cells. Methods of phenotypic sorting include, but are not limited to, confocal microscopy, flow cytometry, Western blotting, RT-PCR, IB4 lectin binding and co-enrichment. It is understood that not all methods of phenotypic sorting are suitable for all genetic target modifications. Counter-selection with IB4 lectin binding is particularly useful for modifications of the αGal gene. Further counter-selection with IB4 lectin binding is particularly useful for multiple modifications, when at least one target is the αGal gene. In summary, xenoantigens αGal, Neu5Gc and a SLA were reduced by genetic modification. Transgenic products were produced within 5-10 months or less.

In embodiments of the present invention, cells are provided in which the αGal and CMAH genes and a SLA gene are rendered inactive, such that the resultant products can no longer generate alpha 1,3-galactosyl epitopes or Neu5Gc on the cell surface and have a reduced level of SLA epitopes on the cell surface. In an alternative embodiment, the αGal, CMAH and SLA genes can be inactivated in such a way that no transcription of the gene occurs. In an embodiment, cells are provided in which alpha-Gal and a SLA gene are rendered inactive and the cells express an HLA product.

In yet another aspect, the present invention provides a method for producing viable pigs lacking any functional expression of αGal, SLA and CMAH. In one embodiment, the pigs are produced as described below. Methods of making transgenic pigs, and the challenges thereto, are discussed in Galli et al. 2010 Xenotransplantation, 17(6) p. 397-410, incorporated by reference herein for all purposes. The methods and cell cultures of the invention are further detailed below.

The following Examples are offered for illustrative purposes only and are not intended to limit the scope of the present invention in any way. Indeed various modifications in addition to those shown and described herein will become apparent to those skilled in the art from the foregoing description and the following examples and fall within the scope of the appended claims

### EXAMPLES

### Example 1

### Design of Targeting Vectors

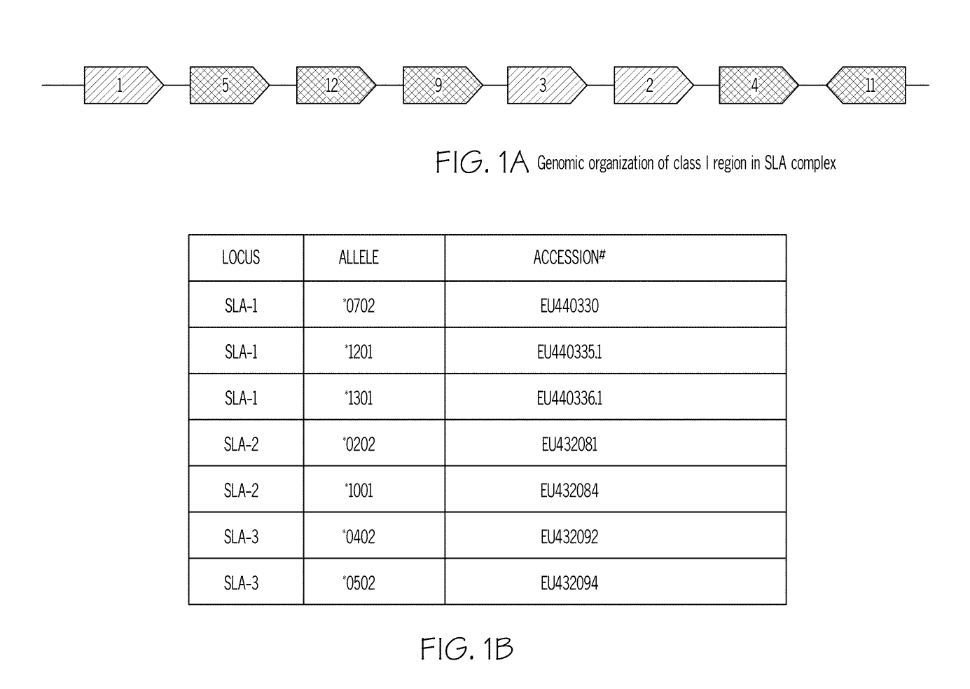

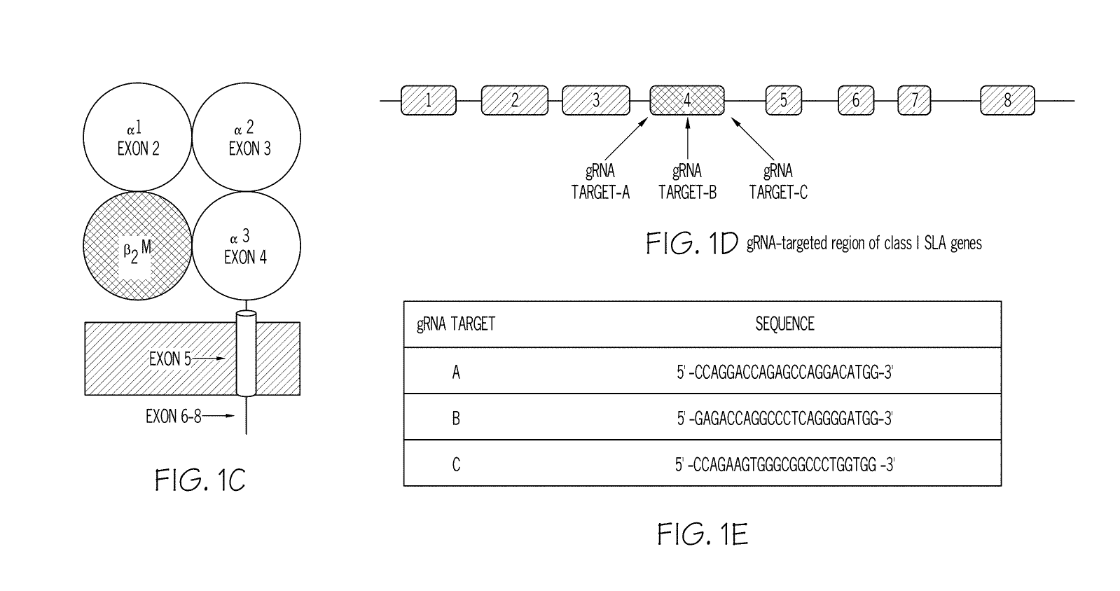

A CMAH Crispr construct with a sequence that is the reverse complement of a portion of the sequence listed in Ensemble transcript ENSSSCT00000001195 was created and utilized in the creation of a double transgenic product. A Gal Crispr construct with a sequence identical to a portion of that in the appropriate Ensemble transcript ENSSSCT00000006069 was created and utilized in the creation of a double transgenic product. Three SLA CRISPR constructs with sequences identical to a portion of the SLA Class I region were created and utilized in the creation of a transgenic product. SLA targeting sequences are shown in FIG. 1.

Plasmid pX330-U6-Chimeric_BB_CBh_hSpCas9 (Addgene plasmid 42230) was used to clone the designed annealed oligonucleotides (FIG. 1E) to generate gRNA using the CRISPR-associated Cas9 nuclease system. One microgram pX330 was digested with Bbsl (New England Biolabs, Ipswich Mass.) for 30 minutes at 37° C. Each pair of phosphorylated oligonucleotides was annealed using a Veriti thermocycler (Applied Biosystems, Grand Island N.Y.) starting at 37° C. for 30 minutes, followed by a step at 95° C. for 5 min and then ramp down to 25° C. at 5° C/min. Digested pX330 was ligated to the annealed pair of oligonucleotides for 10 minutes at room temperature. Ligation reaction was used to transform TOP10 competent cells (Invitrogen), following the manufacturer's protocol. The QIAPrep kit (Qiagen Valencia Calif.) was used to isolated plasmid from 15 colonies per treatment. DNA clones were sequenced and used to transfect porcine fetal fibroblasts.

### Example 2

### Production of Transgenic Cells

Fetal fibroblast cells from a cloned pig with known class I SLA alleles were used in this study (See for example Reyes et al (2014), Tissue Antigens 84(5):484-488, herein incorporated by reference in its entirety). Fetal fibroblasts cultured in stem cell media (FFSCs) were resuspended and cultured in MEM-α (Invitrogen, Carlsbad, Calif.)/EGM-MV (Lonza, Basel, Switzerland) media supplemented with 10% FBS (HyClone, Logan Utah), 10% horse serum (Invitrogen), 12 mM HEPES (Sigma-Aldrich, St. Louis Mo.), and 1% penicillin/streptomycin (Life Technologies, Grand Island N.Y.) and cultured in collagen-I-coated plates (Becton Dickinson, Bedford Mass.) at 38.5° C., 5% CO₂ and 10% O₂. The cells treated with SLA- specific gRNA and Cas9 contained a previously inactivated GGTA1 gene. SLA-expressing control cells were derived from GGTA1-deficient animals. The genetic backgrounds of the control and experimental animals are very similar, as they were cloned from cell originating from a single donor.

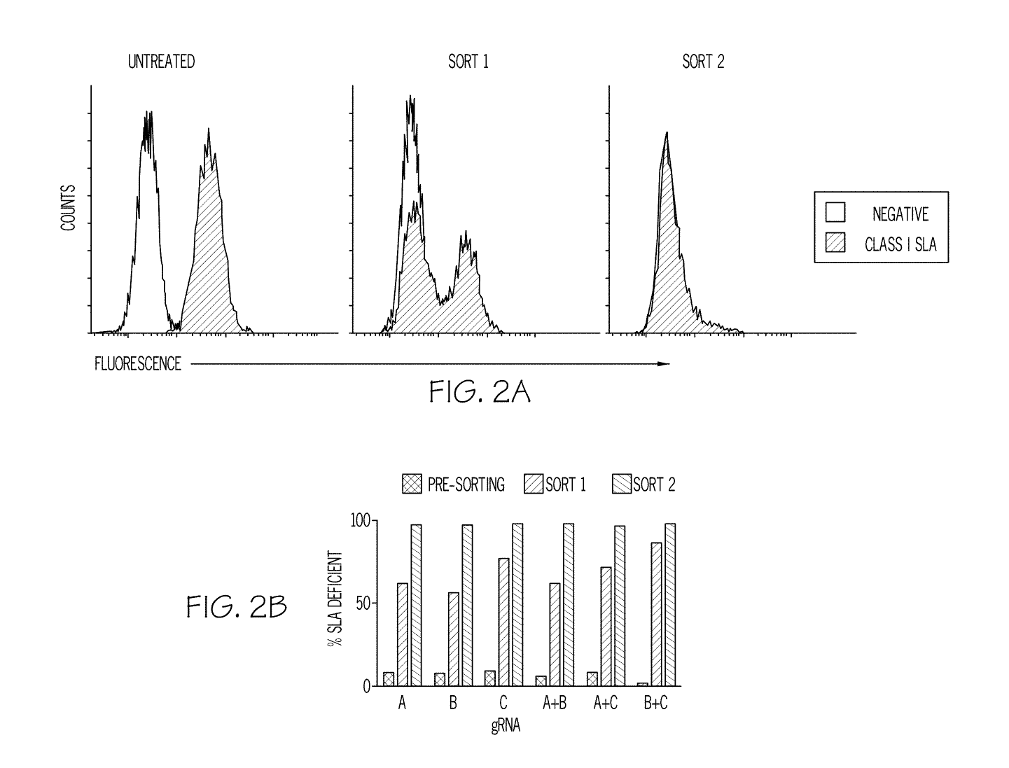

FFSC's were seeded in early passage (passage 2) onto six-well plates 24 hours before transfection. Cells were harvested and counted and 1×10⁶ cells were resuspended in 800 μl fresh sterile electroporation buffer (75% cytosalt buffer: 120 mM KCl, 0.15 mM CaCl₂, 10 mM K₂HPO₄ [pH 7.6], 5 mM MgCl₂) and 25% Opti-Mem (Life Technologies). Cells were mixed with 2 μg plasmid DNA in 4 mm cuvettes. Transfection was performed using the Gene Pulser Xcell (Bio-Rad, Hercules, Calif.) following the manufacturer's recommended protocols for mammalian cells. Treated cells were seeded onto six-well plates and grown until confluent. Cell screening was performed using a BD Accuri C6 flow cytometer (BD Biosciences, San Jose Calif.) using mouse anti-pig SLA class I-FITC Ab (AbD Serotec, Raleigh N.C.). Cells with low expression for SLA class I Ag were expanded and FACS was used at least twice with a FACSVantage SE. Representative results are shown in FIG. 2.

### Example 3

### Flow Cytometry Analysis

For flow cytometry, porcine PBMCs were prepared using Ficoll-Paque Plus as described elsewhere (See Lutz et al, 2013 Xenotransplantation 20:27-35, herein incorporated by reference in its entirety). PBMC were stained with the following Abs: mouse anti-pig PerCP-Cy 5.5 CD3, PE CD4, FITC CD8α and mouse isotype control (BD Biosciences). Dead cells were excluded from analysis using fixable viability dye eFluor 660 (eBioscience, San Diego Calif.). Analysis was performed using an Accuri C6 flow cytometer and CFlow software (Accuri, Ann Arbor Mich.) and FlowJo software (TreeStar, Ashland Oreg.).

Primary kidney endothelial cells were isolated using 0.025% collagenase type IV from Clostridium histolyticum (Sigma-Aldrich) and cultured for 3 days in RPMI 1640 medium supplemented with 10% FBS and 100 μg/ml endothelial cell growth supplement (BD Biosciences).

Fibroblasts were grown under the same conditions used to maintain fetal fibroblasts as described above.

### Example 4

### Evaluation of Response to Transgenic Xenograft

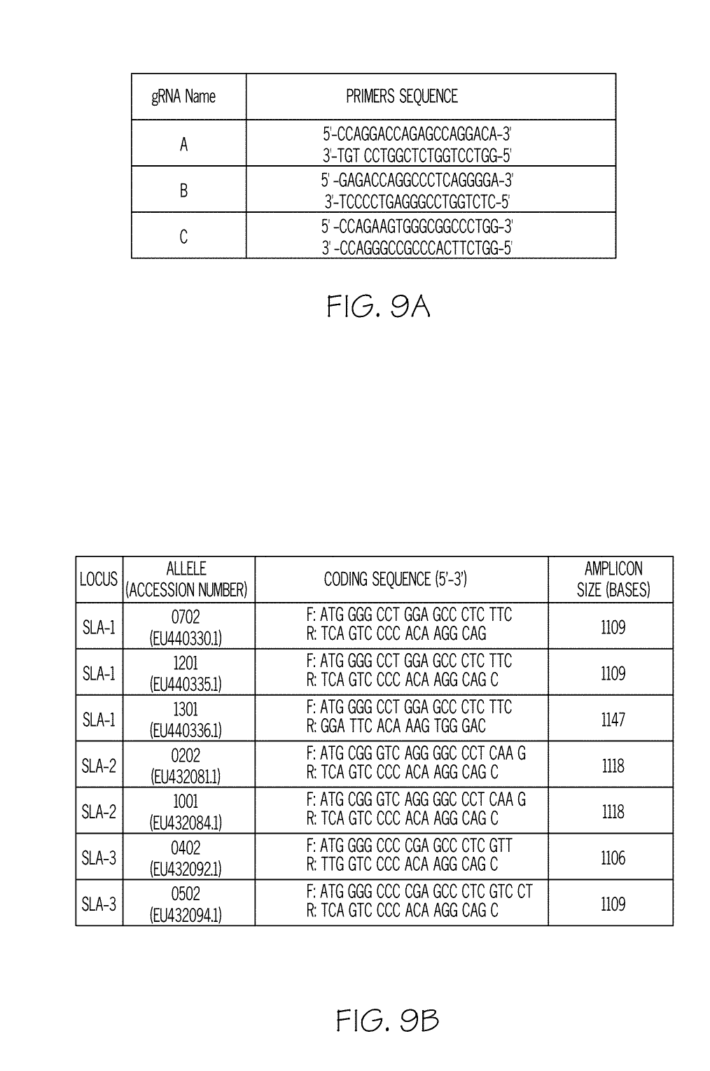

Porcine kidneys were obtained from GGTA1−/−/hDAF transgenic pigs. hDAF is also known as CD55 Five rhesus macaques received donor kidneys following bilateral nephrectomy. Anti-pig IgG antibody titers were determined by flow cytometry analysis (xenograft crossmatch assay using GGTA1-/1 cells as targets) prior to transplant. Four of the five animals had low anti-pig IgG titers. T cells were depleted by treating the recipient monkeys with one dose anti-CD4/anti-CD8. Costimulation blockade was performed using either Fc-intact anti-CD154 (5C8, n=3) or belatacept (n=2). Monkeys were treated with MMF and steroids as well. The monkey with a high titer of non-Gal antibody exhibited excellent initial graft function and normal platelet counts but developed acute humoral rejection with profound thrombocytopenia (platelet count dropped from above 300,000 to less than 10,000), graft interstitial hemorrhage, and significant IgG and IgM deposition on glomerular capillaries. The two monkeys with low initial anti-pig IgG titers treated with anti-CD154 maintained normal renal function platelet counts up to at least 35 days post transplant. Of the two monkeys treated with belatacept, both maintained normal platelet counts but one monkey rejected the graft at post-operative day 14. The second belatacept treated monkey exhibited elevated creatinine post-transplant and histology indicated a T-cell infiltrate with arteritis, consistent with rejection. Results from one such experiment are shown in FIG. 9.

### Example 5

### SLA Class I −/− Pigs

A SLA class I locus was targeted for genome editing. Porcine fetal fibroblasts were transfected with sgRNA targeted to SLA1 and Gal as described above herein according to the manufacturer's instructions. Selected treatments were used for SCNT.

SCNT was performed using in vitro matured oocytes (De Soto Biosciences Inc, St. Seymour Tenn. and Minitube of America (Mount Horeb, Wis.) as described in Estrada et al (2007) Cloning Stem Cells 9:229-236, herein incorporated by reference. Cumulus cells were removed from the oocytes by pipetting in 0.1% hyaluronidase. Oocytes with normal morphology and a visible polar body were selected and incubated in manipulation media (calcium-free NCSU-23 with 5% fetal bovine serum (FBS) containing 5 μg/ml bisbenzimide and 7.5 μg/ml cytochalasin B for 15 minutes. Following this incubation period, oocytes were enucleated by removing the first polar body and metaphase II plate. Single cells of site targeted SLA class I−/− cells were injected into each enucleated oocyte. Electrical fusion was induced with a BTX electroporator (Harvard Apparatus, Holliston Mass.). Enucleated oocytes injected with a cell (couples) were exposed to two DC pulses of 140 V for 50 μs in 280 mM mannitol, 0.001 mM CaCl₂ and 0.05 mM MgCl₂. After activation the oocytes were placed in NCSU-23 medium with a 0.4% bovine serum albumin (BSA) and incubated at 38.5° C., 5% CO₂ in a humidified atmosphere for less than one hour. Within an hour after activation, oocytes were transferred into a recipient pig. Two hundred eleven cloned embryos were transplanted into two recipient pigs.

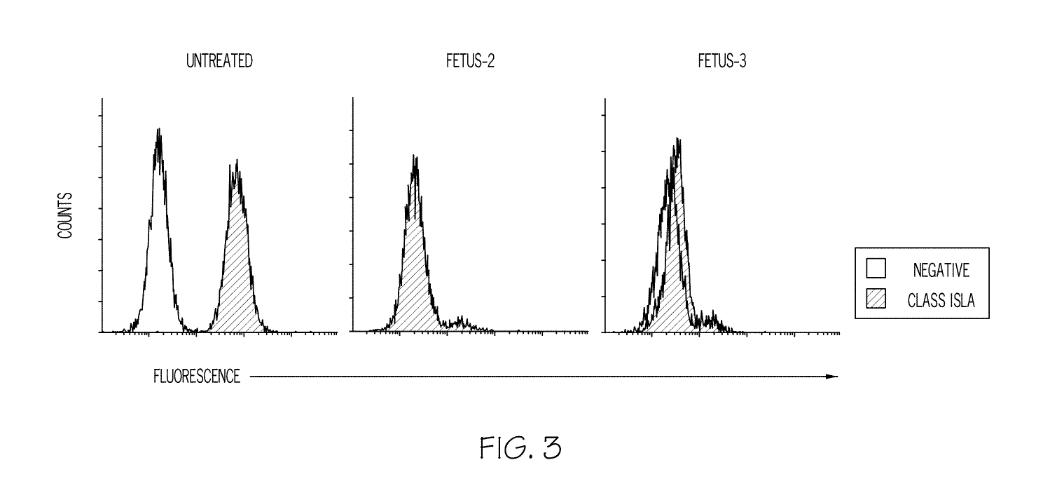

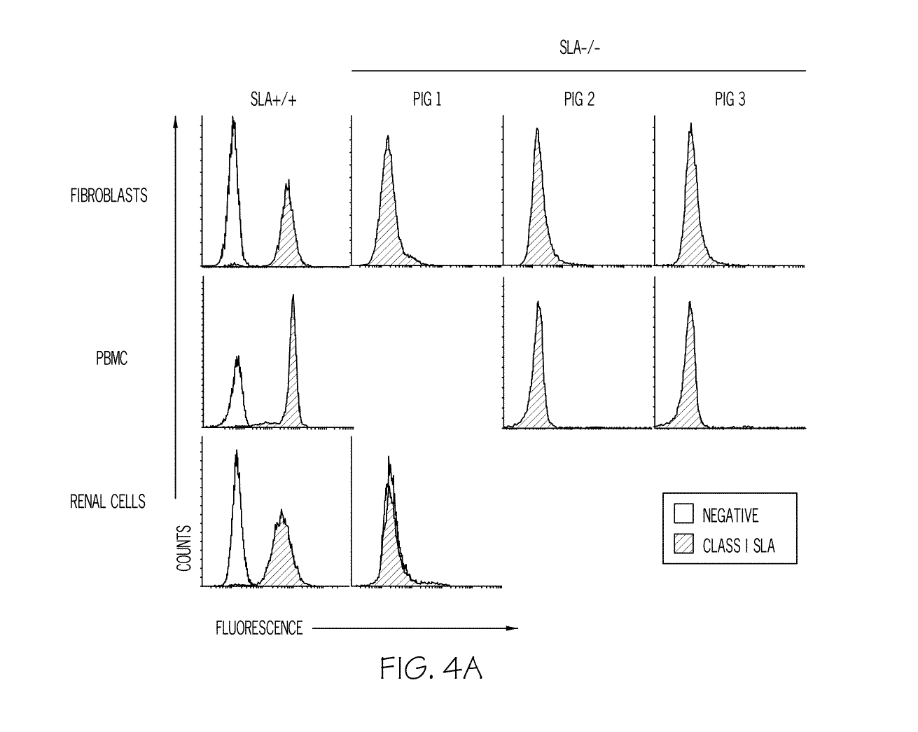

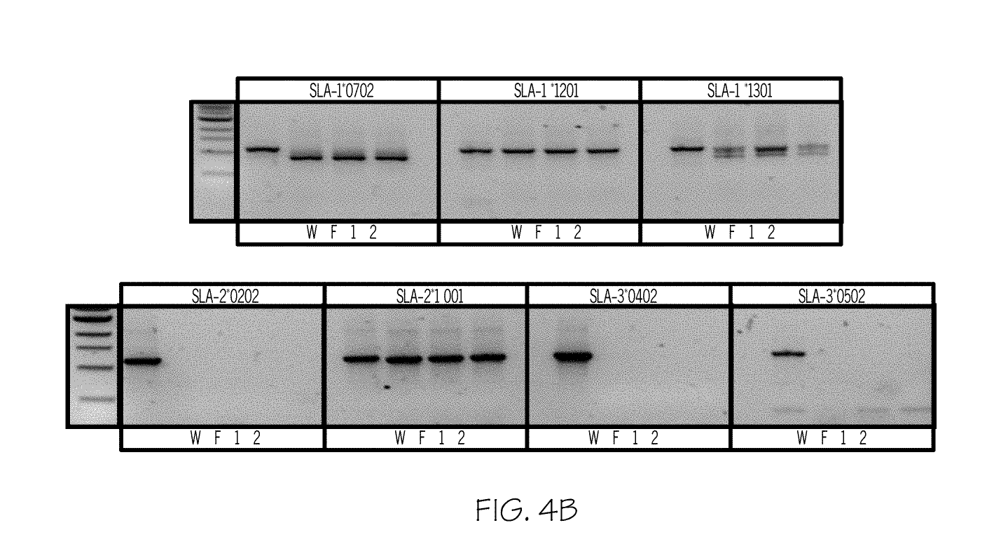

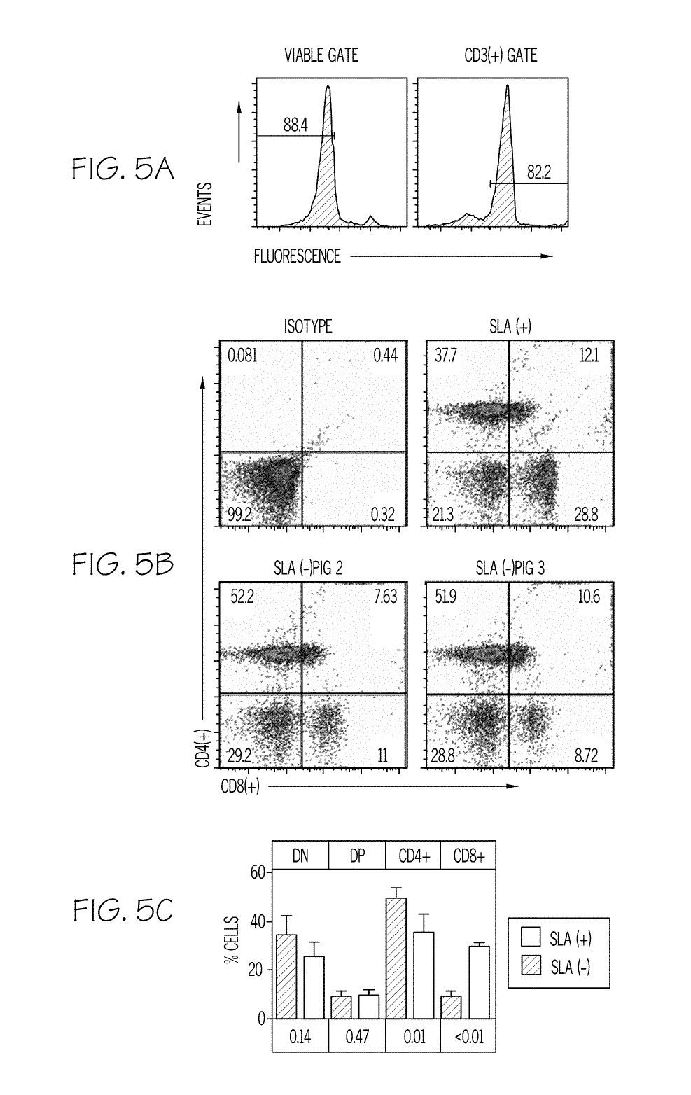

Recipient pigs were synchronized occidental pigs on their first day of estrus. One of the pigs became pregnant. Pregnancies were verified by ultrasound approximately day 25 or day 26 after embryo transfer. Thirty-two days after embryo transfer, three fetuses were collected. Two fetuses were well-formed and used to create fibroblast cultures. The fibroblasts were stained with a negative isotype control or with an antibody specific for class I SLA. Results from one such experiment are shown in FIG. 3. Fetus 2 cells remained negative for SLA class I expression even after recloning. Cells from fetus 2 were used to produce two additional pregnancies. One pregnancy spontaneously terminated at day 45; the other pregnancy produced three viable clonal piglets. The class I−/− pigs were viable and appeared healthy. One animal was sacrificed for cell collection at 1 week of age. Flow cytometry analysis of renal cells obtained from piglet one, PBMC's from piglets 2 and 3 and fibroblasts from all three SLA Class 1−/− pigs are presented in FIG. 4A. See for example Reyes et al, 2014 J. Immunol. 193:5751-5757, herein incorporated by reference in its entirety.

### Example 6

### Genotype Analysis of SLA Class I−/− Products

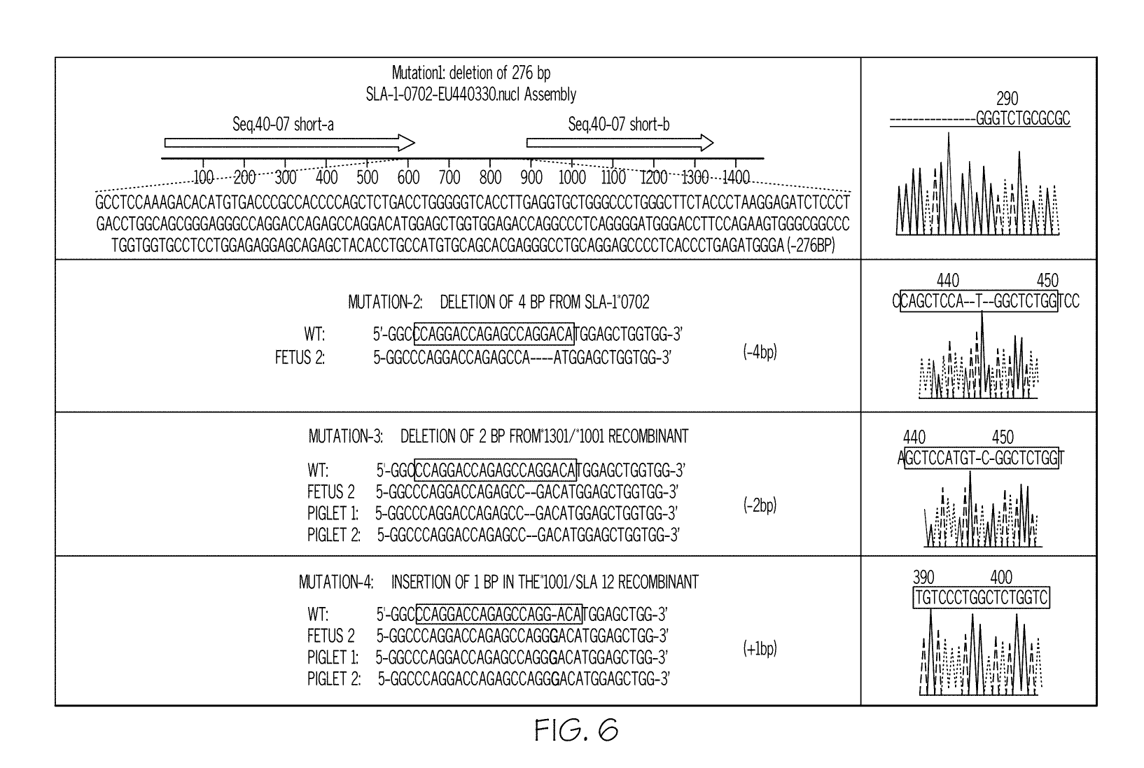

Genomic DNA was isolated from pig cells using the Qiamp DNA minikit (Qiagen). RNA samples were isolated using the RNeasy Plus mini kit (Qiagen) following the manufacturer's protocol. RNA quality and quantity were affirmed by Agilent bioanalyzer analysis. RNA samples were reverse transcribed using a OneStep RT-PCR kit. PCR products were purified and ligated into the pCR4-TOPO TA (Invitrogen). Transformed bacteria were plated on Luria-Bertani agar containing 50 μg/ml kanamycin for clone selection. Plasmids were isolated using the QIAprep Spin Miniprep kit (Qiagen). Nucleotide sequences were performed by the Sanger method using custom sequencing service and the primers indicated in FIG. 9. SLA Class I−/− piglets contained a variety of mutations including a 276 bp deletion that eliminates the α3 domain of the wildtype protein, a 4 base pair deletion that creates a frameshift mutation, and recombinations between various alleles. The 276 bp deletion and 4 base pair deletion were in the SLA 1*0702 allele. Recombinant mutations included SLA-1*1301 and SLA-2*1001, SLA-1*1301 and SLA1*0702, and SLA2*1001 and SLA12 recombination events near the gRNA binding sites. The recombinants molecules were incapable of encoding functional class I SLA molecules as a consequence of frameshifts arising from a 2 base deletion or a 1 base insertion. The mutations are summarized in FIG. 6.

### Example 7

### SLA Class II−/− Fetal Fibroblasts

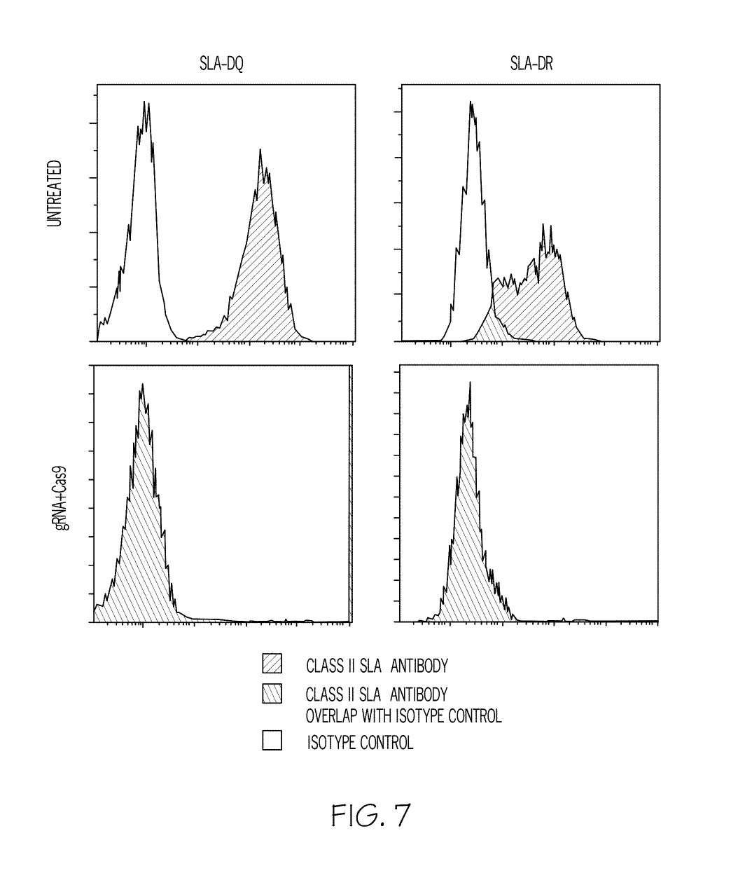

Primary swine fetal fibroblasts were obtained. The fetal fibroblasts were treated with gRNA specific for SLA-DQ or SLA-DR. SLA-DQ and SLA-DR are class II SLA molecules. Cells not treated with gRNA and Cas9 were used as positive controls. Transfected and untreated cells were treated with interferon γ to induce expression of SLA Class II molecules. Expression of SLA class II in interferon γ treated cells was analyzed by flow cytometry. Results from one such experiment are shown in FIG. 7.

### Example 8

### Analysis of Xenoantigen Impact on Antibody Binding

GGTA−/−, SLA Class 1−/− double transgenic pigs have been made. A corresponding double transgenic immortalized renal endothelial cell line is produced from the double transgenic pigs. The double transgenic immortalized renal cell line is used as the background for testing of additional gene deletions. Each triple transgenic cell line is assessed using the flow-cytometry based xeno-crossmatch assay to quantify the impact of deleting the additional gene on antibody binding. Sera from several rhesus macaques (n=10) are used to generate an antibody binding profile. A cell line lacking only the GGTA1 and SLA class I is used as a control. Changes in antibody binding are analyzed using a paired t-test.

### Example 9

### IB4 Counterselection of Triple Transgenics

Cells are transfected with three sets of targeting constructs (αGal, SLA and CMAH). Cells are selected with IB4, a substance that binds αGal. The bulk population of cells that survive IB4 counterselection are used directly in SCNT to make pregnant pigs. Fetuses are collected and analyzed. Fetal fibroblasts are obtained from one such fetus and used in SCNT.

### Example 10

### Production of Transgenic Pigs (Triple)

Somatic cell nuclear transfer (SCNT) is performed using in vitro matured oocytes (DeSoto Biosciences Inc., St. Seymour Tenn. and Minitube of America (Mount Horeb Wis.). Cumulus cells are removed from the oocytes by pipetting in 0.1% hyaluronidase. Oocytes with normal morphology and a visible polar body are selected and incubated in manipulation media (calcium-free NCSU-23 with 5% fetal bovine serum (FBS) containing 5 μg/ml bizbenzimide and 7.5 μg/ml cytochalasin B for 15 minutes. Following this incubation period, oocytes are enucleated by removing the first polar body and metaphase II plate. For the triple transgenic pigs, single cells of site-targeted liver derived cells (LDC) that survive IB4 counterselection are injected into each enucleated oocyte. Electrical fusion is induced with a BTX electroporator (Harvard Apparatus, Holliston Mass.). Enucleated oocytes injected with a cell (couples) are exposed to two DC pulses of 140 V fo 50 μs in 280 mM mannitol, 0.001 mM CaCl₂ and 0.05 mM MgCl₂. After activation the oocytes are placed in NCSU-23 medium with 0.4% bovine serum albumin (BSA) and incubated at 38.5° C., 5% CO₂ in a humidified atmosphere for less than one hour. Within an hour after activation, oocytes are transferred into a recipient pig. Recipient pigs are synchronized occidental pigs on their first day of estrus. Pregnancies are verified by ultrasound at day 25 or day 26 after embryo transfer. Fetal fibroblasts are taken from one triple transgenic fetus for SCNT. Other pregnancies are allowed to culminate in the production of viable liters of genetically modified pigs.

All animals used in this study are approved by the Institutional Biosafety Committee (IBC) and Institutional Animal Care and Use Committee (IACUC).

### Example 11

### Ex Vivo Perfusion of Human Platelets Through Transgenic Liver

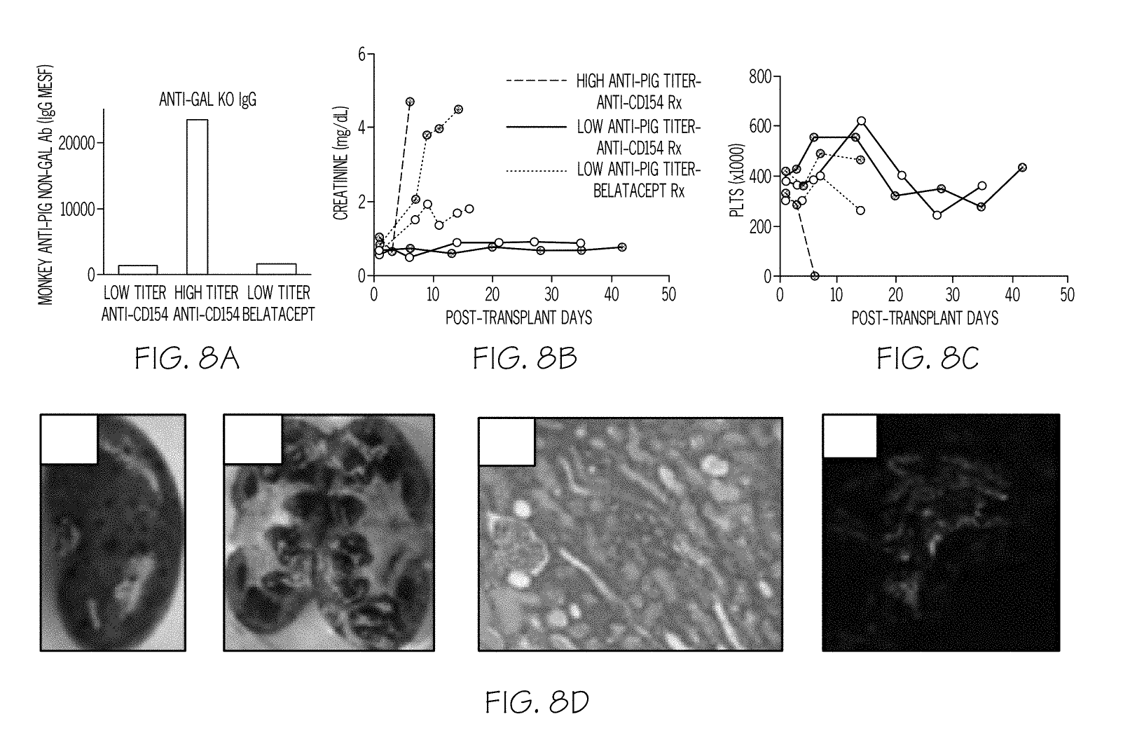

A triple transgenic GGTA1/CMAH/SLA pig is anesthetized and intubated. A midline abdominal incision is made. The liver is removed and placed in a perfusion device under normothermic conditions. A continuous perfusion circuit contains a heated buffer reservoir, three pumps (1, continuous venous return; 2 pulsatile arterial supply; 3 continuous portal vein supply), an oxygenator (O₂), two bubble traps (BT) and flow (F) and pressure (P) monitors. The system is computer controlled to maintain perfusion with specific parameters. A diagram of an ex vivo perfusion device is shown in FIG. 8. Humidity, temperature and air flow are maintained in the perfusion device. The perfusion device maintains constant pressure by varying the flow rate. Centrifugal flow through the portal vein and pulsatile flow through the hepatic artery are used. Both flow rates are set at porcine physiological pressure. The base perfusion solution is an oxygenated Ringers solution with physiologic nutrition and insulin.

Human platelets are obtained from healthy volunteer subjects or purchased commercially less than six days from isolation and are stored at 20-24° C. Approximately 1×10¹¹ human platelets are washed in sterile phosphate buffered saline (PBS) containing the anti-coagulant citrate dextrose. Platelets may be labeled with CFSE according to the manufacturer's protocol.

Pig livers are perfused two hours prior to the addition of platelets. Platelet samples are obtained prior to addition to the perfusion system and after the addition of the platelets at pre-determined time points. Platelet levels in the pre-perfusion and post-perfusion samples are evaluated. Pre and post-perfusion evaluation of the pig liver are performed. Wild-type pig livers are obtained, and the livers are perfused under similar conditions.

### Example 12

### Evaluation of Response to a Transgenic Xenograft

Porcine livers are obtained from a triple transgenic pig (αGal, CMAH, SLA). The livers are surgically transplanted into a recently deceased human cadaver using the piggyback method. After the surgery, biological samples are obtained from the human cadaver. Clinical indicia of a rejection related response are monitored.

### Example 13

### Evaluation of Response to a Transgenic Xenograft

Porcine kidneys are obtained from a triple transgenic pig (αGal, CMAH, SLA). A highly sensitize human subject is administered compounds to manage preexisting and de novo donor-specific antibodies. The porcine kidneys are surgically transplanted into the subject. After the surgery, biological samples are obtained from the human cadaver. Clinical indicia of a graft rejection are monitored.

### Example 14

### Confocal Microscopy Analysis

Piglets (triple GGTA1, CMAH, SLA transgenics, wild type or other piglets of interest) are euthanized. Liver, heart and kidney tissue are obtained from the pig. Frozen sections of each tissue are prepared. Mounted tissues are blocked in Odyssey blocking buffer (Li-Cor Biosciences, Lincoln Nebr.) in HBSS for one hour. The slides are fixed in 4% paraformaldehyde for 10 minutes. Tissues are stained with IB4 lectin Alexa Fluor 647 (Invitrogen, Grand Island N.Y.) to visualize the presence of the Gal epitope. To visualize the Neu5Gc epitope, tissues are stained with a chicken anti-Neu5Gc antibody or with a control antibody (Sialix, Vista Calif.) for an hour. Tissues are washed three times with HBSS. Donkey anti-chicken Dylight 649 (Jackson ImmunoResearch Laboratories Inc, West Grove Pa.) secondary antibody is incubated with the tissue for approximately an hour. Tissues are washed three times with 0.1% HBSS Tween. To stain the nucleus, DAPI stain (Invitrogen, Grand Island N.Y.) is added to all the slides for 1 minute followed by two 0.1% HBSS Tween washes. Tissues are mounted in ProLong Gold (Invitrogen, Grand Island N.Y.). Confocal microscopy is performed using an Olympus FV1000.

### Example 15

### Crossmatch of Human Sera with Transgenic PBMCs

Porcine whole blood from transgenic (triple GGTA-1/SLA/CMAH for example) and wild-type pigs are collected in ACD. Porcine peripheral blood monocytes (PBMCs) are prepared from the whole blood using Ficoll-Paque Plus. Cell viability is assessed microscopically with Trypan Blue. Sera are obtained from healthy human volunteers. Twenty-five percent heat inactivated serum is prepared. Approximately 2×10⁶/ml porcine PBMCs are incubated with each human serum sample for two hours at 4° C. After incubation of the serum and PBMCs, the PBMCs are washed three times in 0.5% PBS Sialix Blocking agent. PBMCs are stained with DyLight 649-conjugated donkey anti-human IgM or DyLight 488 donkey anti-human IgG (Jackson Immunoresearch Laboratories Inc., West Grove Pa.) for 1 hour at 4° C. PBMCs are washed three times using 0.5% PBS Sialix blocking agent. Analyses are performed using an Accuri C6 flow cytometer and BD CFlow Plus Software (Accuri, Ann Arbor Mich.). Overlays are produced using Kaluza software from Beckman Coulter (Brea Calif.).

### Example 16

### Antibody-Mediated Complement-Dependent Cytotoxicity

Antibody-mediated complement dependent cytotoxic assays are known in the art. A method of Diaz et al (Diaz et al., 2004 Transplant Immunology 13(4):313-317) is performed. Human serum is obtained from healthy volunteers. Twenty-five percent heat inactivated serum is prepared. Heat-inactivated human sera are serially diluted and 100 μl of each concentration is placed in a 96 well v-bottom assay plate. The sera is mixed with a 100 μl aliquot of PBMC obtained from a pig of interest (GGTA1/CMAH/SLA triple or other). PBMC final concentrations are either 5×10⁶/ml or 1×10⁶/ml. Serum concentrations vary from 50%, 17%, 2%, 0.6%, 0.2%, and 0.07%. The mixtures are incubated for 30 minutes at 4° C. After 30 minutes, the plates are centrifuged for 4 minutes at 400×g. The plates are decanted and washed with HBSS. Rabbit complement (150 μl of a 1:15 dilution) is added to each well and incubated for 30 minutes at 37° C. PBMC are labeled with a fluorescein diacetate (FDA) stock solution, prepared fresh daily in HBSS (1 μg/ml) from a 1 mg/ml stock solution in acetone and with propidium iodide (PI), prepared at 50 μg/ml in phosphate buffered saline (PBS). After incubation in complement, the samples are transferred by pipette to tubes containing 250 μl of HBSS and 10 μl of FDA/PI for analysis using an Accuri C6 flow cytometer.

The percentage of dead cells (PI+/FDA−), damaged cells (PI+/FDA+) and live cells is determined. Double negative events (PI−/FDA−) are excluded from calculations. The percentage of cytotoxicity in cells not exposed to serum is considered spontaneous killing. Values for cytotoxicity are corrected for spontaneous killing.

### Example 17

### Porcine Liver Procurement

Pigs are premedicated, intubated and anesthetized with propofol and placed in the supine position. A midline incision to the abdomen is made. Ligamentous attachments to the liver are taken down. The portal vein and hepatic artery are cannulated and flushed with 2 liters of cold histidine-tryptophan-ketoglutarate solution (Essential Pharmaceuticals, LLC). Livers are removed from pigs and stored in histidine-tryptophan-ketoglutarate solution on ice at 4° C. until being placed in a liver perfusion circuit. Cold-ischemia time varies between 45 minutes to 3 hours. In certain experiments porcine livers may be obtained from abbatoirs. Porcine livers from abbatoirs are flushed with histidine-tryptophan-ketoglutarate solution containing heparin (2000 U/L) within two minutes of exsanguinations.

### Example 18

### Assessment of Impact of Expression of Rhesus Class I and II

Porcine cells expressing MamuA01 as described above herein are evaluated for an impact on T cell proliferation and NK and T cell mediated cytotoxicity using standard assays.

### Example 19

### Assessment of Gene Deletion on the Cellular Immune Response to Xenograft

Cells are obtained from class I, class II or combined class I/II gene transgenic pigs. The T-cell proliferative response is assessed in vitro using a flow-cytometry based CFSE MLR assay. The T-cell mediated cytotoxicity of combined class I/II transgenic pig cells are evaluated in flow-based T cell cytotoxicity assays using CD107a as a measurement of degranulation and vital dyes to detect pig target cell killing. See Chan & Kaur. 2007 J. Immunol Methods 325:20-34 and Kitchens et al (2012) Am J. Transplant 12:69-80, herein incorporated by reference in their entirety. Cytolytic activity is calculated based on the percent of CD107a+ cells; cell subsets are characterized as CD4+ and CD8+ T cells, CD3-CD16+ NK cells. Proliferation and killing assay results are considered when deciding which deletion pigs to use as pig donors.

### Example 20

### Kidney Xenograft in NHP

Recipient non-human primates (NHP) are treated with one dose of anti-CD4/anti-CD8, anti-CD154/anti-CD28 dAbs, MMF and steroids. Rhesus macaques (Macaca mulatta) are used as the NHP. In some experiments, macaques may be 3-5 years old and less than 6 kg. Transgenic porcine kidneys (or wildtype control kidneys) are transplanted into the NHP recipients. Samples (blood, urine and kidney biopsy samples) are collected at defined time points for analysis. Renal function, serum creatinine, the presence and quantity of xenoantibodies (flow cytometry and multi-parameter flow cytometry), cytokine secretion, transcript profiles from peripheral blood, urine and graft biopsies, xenograft histology and development of anti-pig antibody (flow-based xenocrossmatch assay) are followed. The CMAH deletion is not helpful for study in NHP. For NHP studies, pigs with a wild-type CMAH gene are used. Ultrasound guided needle biopsies are performed at 2, 5 and 10 weeks post transplant.

### Example 21

### Veterinary Care of NHP

NHP are housed in individual cages and provided with clean, adequately sized living quarters; fed twice daily; and are checked at least twice daily by animal care technicians and once daily by clinical veterinary staff. Physical examinations are performed each time an animal is anesthetized for blood collection or other procedures.

### Example 22

### NHP Phlebotomy and Tissue Sampling

Phlebotomy and tissue sampling (for example: blood collections, lymph node biopsies and bone marrow aspirates) of NHP's are performed either under ketamine (10 mg/kg) or Telazol (4 mg/kg) anesthesia on fasting animals. Buprenephrine (0.01 mg/kg every 6 hrs) is administered as post-operative analgesia for animals undergoing renal transplant and as needed as determined by the attending veterinarian. Animals are monitored for “irreversible critical illness” such as but not limited to loss of 25% of body weight from baseline; complete anorexia for 4 days; major organ failure or medical conditions unresponsive to treatment such as respiratory distress, icterus, uremia, intractable diarrhea, self-mutilation or persistent vomiting, and surgical complications unresponsive to immediate intervention: bleeding, vascular graft/circulation failure, infection and wound dehiscence.

### Example 23

### Porcine Embryo Transfer Surgery, Phlebotomy and Harvesting Procedures

Embryo transfer surgery: Before surgery, the sow is anesthetized with TKX (Telazol (500 mg)+Ketamine (250 mg) and Xylazine (250 mg); 1 cc per 50 lbs, IM) for intubation plus isoflurane by inhalation through ET tube using a precision vaporizer and waste gas scavenging. During the recovery period, animals are monitored at least once every 15 minutes and vital signs (temperature, heart rate, respiration rate and capillary refill time) are assessed and recorded. Trained animal care technicians or veterinarians monitor the animals until they can maintain themselves in voluntary sternal recumbrance. Animals are returned to regular housing areas upon approval by the attending veterinarian. Post-operative analgesics include buprenorphine 0.01-0.05 mg/kg IM every 8-12 hours or carprofen 2-4 mg/kg SC daily. Approximately 26 days after embryo transfer, ultrasound is performed to confirm establishment of pregnancy while the sow is distracted by food. About 10 days later a second ultrasound is performed. Birth occurs through natural parturition unless clinical difficulty arises. Caesarian section is performed recommended by the veterinary staff. Standard caesarian section protocols are used with the general anesthesia protocol utilized in the embryo transfer surgery. Experimental piglets are cleaned and the umbilical cord is disinfected. Every piglet receives colostrum during the first hours after birth. Piglets are watched 24/7 until they are at least 7 days old. Farrowing crates are used to protect the piglets from their mother while maintaining the piglets ability to nurse.

All phlebotomy is performed under either ketamine (10 mg/k) or Telazol (4 mg/kg) anesthesia on fasting animals. Organ harvesting, a terminal surgical procedure, uses the anesthesia protocol (Telazol (500 mg)+ketamine (250 mg)+xylazine (250 mg); 1 cc per 50 lbs; IM)+/− pentothal (10-20 mg/kg) IV if needed for intubation and isoflurane by inhalation through ET tube using a precision vaporizer, to effect with waste gas scavenging. Swine are perfused with saline followed by removal of the heart and other tissue/organs. Alternatively swine are anesthetized with inhaled anesthetic and treated with a barbituric acid derivative (100-150 mg/kg) and a bilateral pneumothorax is performed.

### Example 24

### SLA Class I and II−/− Pigs

Fetal fibroblasts are obtained from GGTA1−/− (αGal null) swine. sgRNA and Cas9 are used to target SLA class I and class II genes (SLA-1, SLA-2, SLA-3, SLA-DQ or SLA-DR) in the GGTA1−/− fetal fibroblasts. In some embodiments fetal fibroblasts are obtained from wildtype swine; sgRNA and Cas9 are used to target SLA class I, class II genes (SLA-1, SLA-2, SLA-3, SLA-DQ or SLA-DR) and GGTA1. Wildtype fetal fibroblasts treated with sgRNA and Cas9 targeted to GGTA1 are counter-selected for lectin binding. Transfected nuclei are transferred into enucleated oocytes and implanted in a receptive sow. In some instances, fetuses are harvested after thirty days. Cells are isolated from well-developed fetuses, amplified and directly used in re-cloning to generate cloned animals. Some amplified fetal cells are frozen and stored in liquid nitrogen. T cell xenoreactivity will be assessed through assays such as but not limited to the CFSE MLR assay. In the CFSE MLR assay, PBMC from rhesus macaques are incubated with PBMC's from pigs of the indicated genetic background. Dilution of CFSE will assess proliferation in CD4+ and CD8+ T cell subsets. T-cell proliferation inhibitors may or may not be used in CFSE MLR assays.

### Example 25

### Liver Xenograft in NHP

Rhesus macaques are treated with either an anti-CD28 dAb based immunosuppressive regimen (T cell depletion using anti-CD4/anti-CD8, single dose), anti-CD28 dAb, MMF and steroids; an anti-CD154 dAb based immunosuppressive regimen (T cell depletion using anti-CD54/anti-CD8, single dose, anti-CD154 dAb, MMF and steroids) or both regimens. Livers from transgenic pigs are transplanted into rhesus macaques treated with the indicated immunosuppressive regimen. Liver biopsies are performed at 1 hour, 1 week, 4 weeks and at times of liver graft dysfunction. Laboratory assessments include CBC with platelet count and coagulation studies; liver function tests are obtained at 6 hours after transplant, every other day for 1 week and then twice weekly as well as at any time of clinical deterioration. Rejection is characterized using standard diagnostic criteria and immunohistochemistry. NHPs express CMAH, thus the CMAH deletion is not helpful in NHP studies. Some NHP studies are performed with wildtype CMAH pig tissues.

### Example 26

### Transgenic Pigs Expressing PD-L1, Rhesus Class I Molecules or CD47 Molecules

PD-L1 is the ligand for PD-1 (programmed death-1), a potent T cell co-inhibitory molecule. The rhesus PD-L1 gene is used with sgRNA and Cas9 to generate αGal−/−, ASGR1−/−, PD-L1 and αGal−/−, ASGR1−/−, CMAH−/−, PD-L1 expressing pig LSEC's and transgenic αGal−/−, ASGR1−/−, PD-L1 and αGal−/−, ASGR1−/−, CMAH−/−, PD-L1 expressing pigs. The rhesus PD-L1 gene is used with sgRNA and Cas9 to generate αGal−/−, SLA−/−, PD-L1 expressing pig LSEC's and transgenic αGal−/−, SLA−/−, PD-L1 expressing pigs. Mamu A01 and Mamu E (Mamu-E-HLA-E) are two rhesus class I molecules. αGal−/−, ASGR1−/−, and either Mamu A01, Mamu E, or both pigs are created. The human CD47 (hSIRPα) is used with sgRNA and Cas9 to create αGal−/−, SLA−/−, CD47 expressing pigs. Transgenes to be expressed are cloned behind appropriate promoters such as, but not limited to, RSV, CMV, elF-1α or class I MHC promoters and are flanked by endogenous swine DNA sequences. The flanked transgenes are introduced into cells and simultaneously treated with CRISPR/Cas9. The DNA regions flanking the gRNA binding sites are amplified by PCR. The PCR products are cloned into vectors and sequenced.

### Example 27

### Transfection of SLA I Alleles and Flow Cytometry Analysis

On the day prior to transfection, renal endothelial cells were plated on attachment factor (Gibco Life Technologies) in six-well cluster culture plates in RPMI-1640 supplemented with 10% v/v heat-inactivated FBS, 100 micrograms/mL endothelial cell growth supplement (Corning Life Sciences), and 10 mM HEPES. Cells were allowed to recover for 24 hours prior to transfection. The following day, cells were washed and fresh culture media was replaced four hours prior to transfection. DNA was complexed for transfection using Lipofectamine 2000CD (Invitrogen) using 2 micrograms of DNA per well at a ratio of 2:1 of microliters of lipid to micrograms of DNA. Cells were transfected at 80-90% confluency per manufacturer instructions. Cells were then subcultured until flow cytometric analysis.

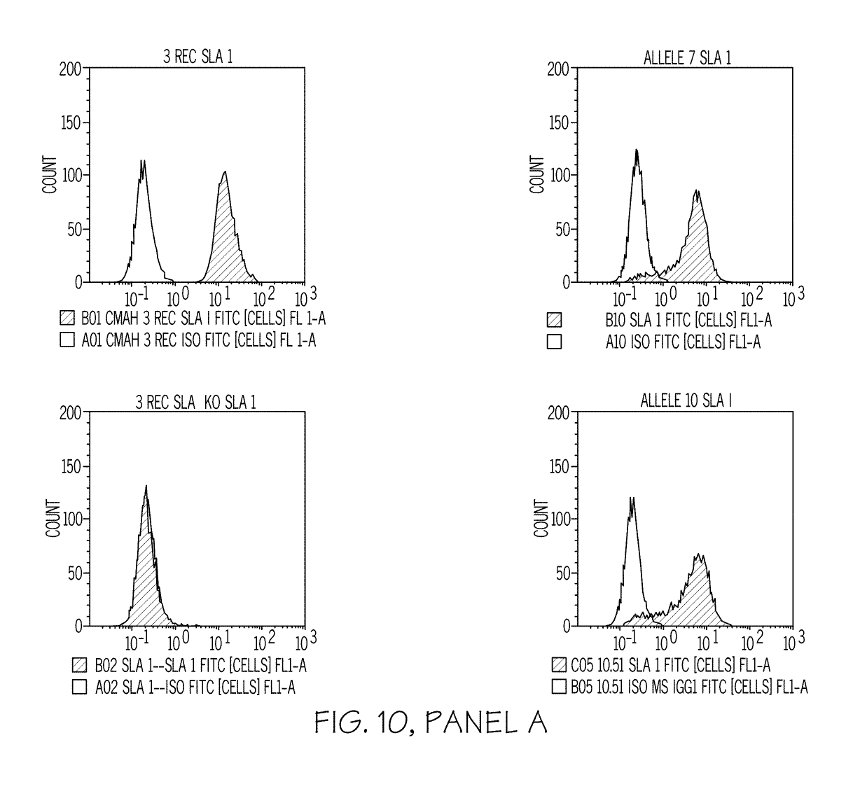

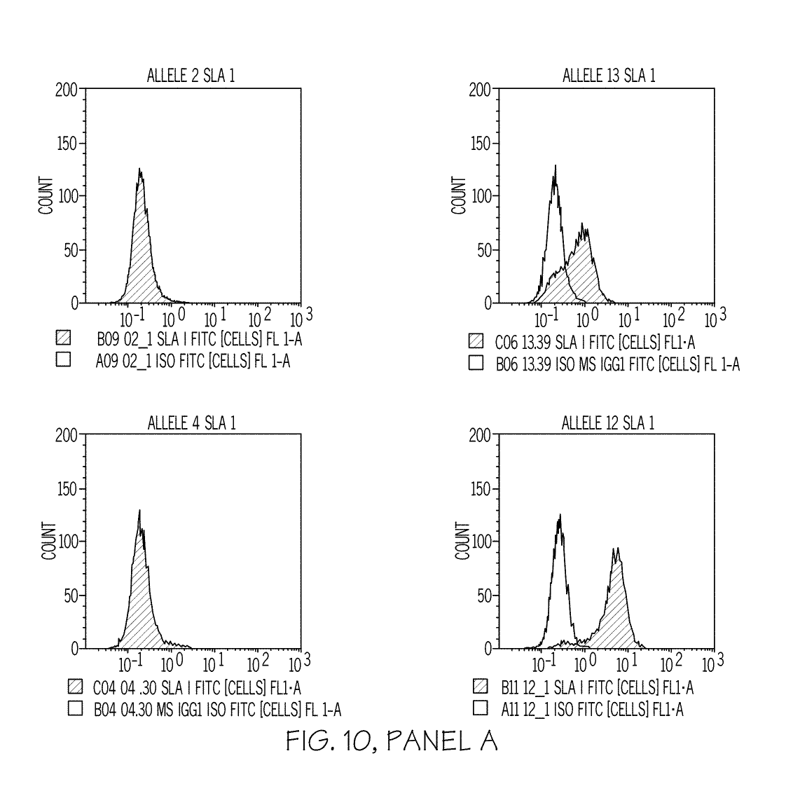

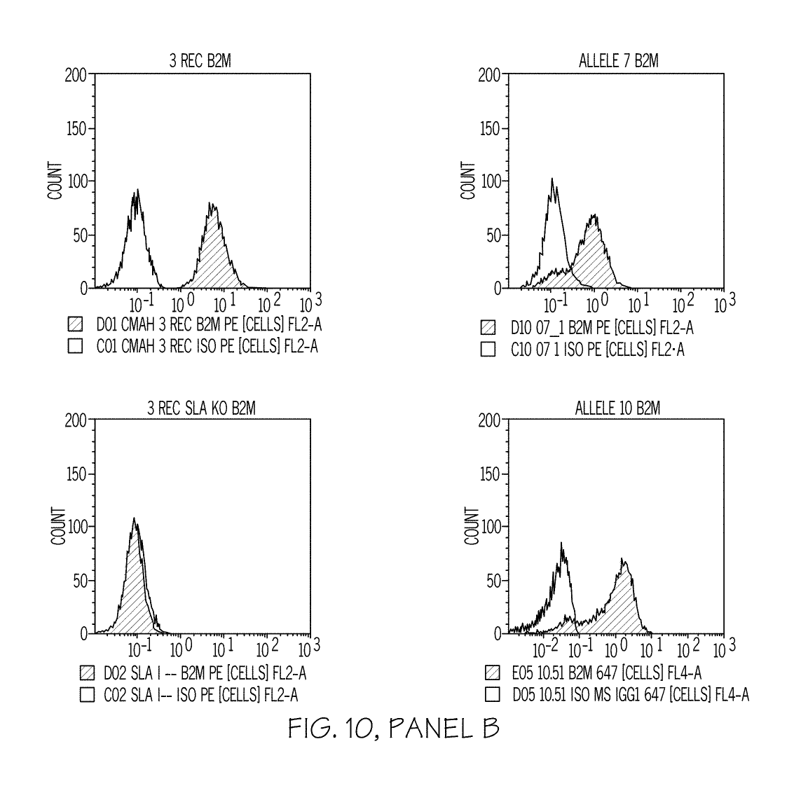

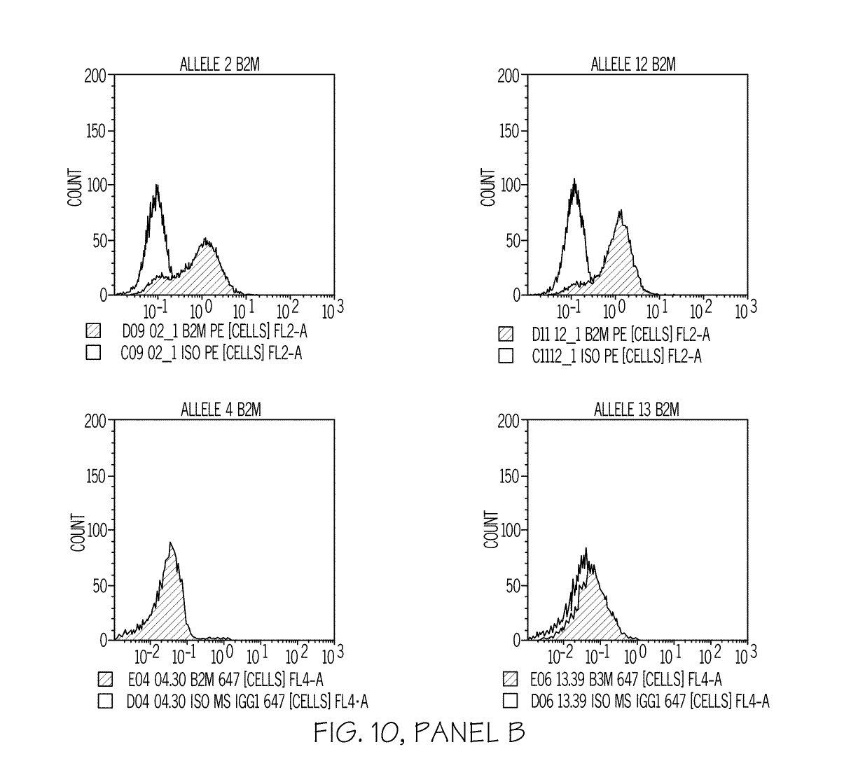

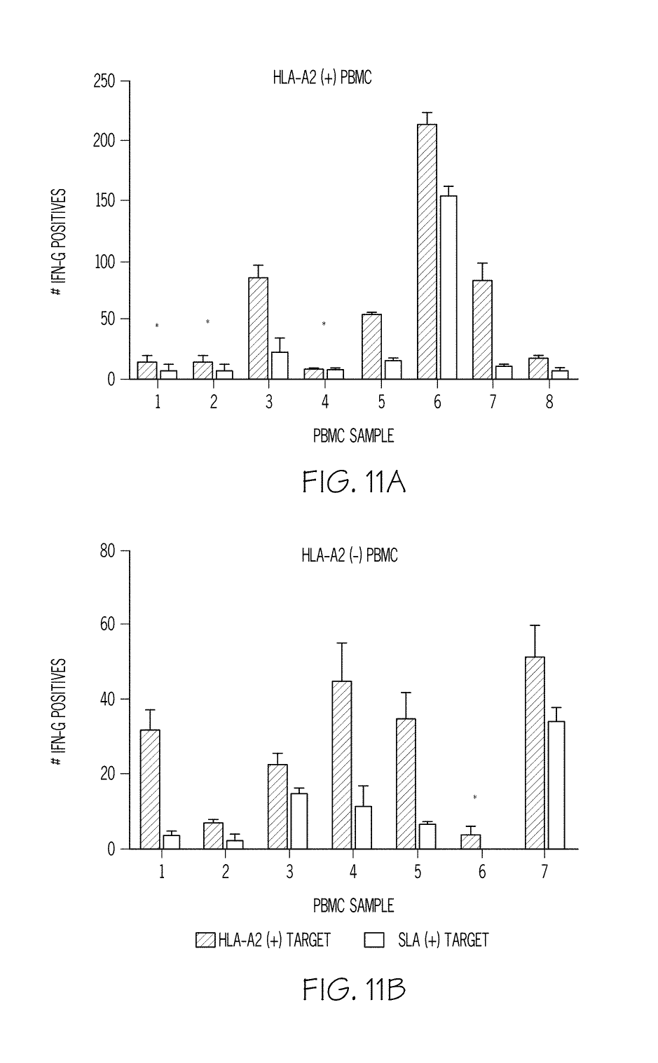

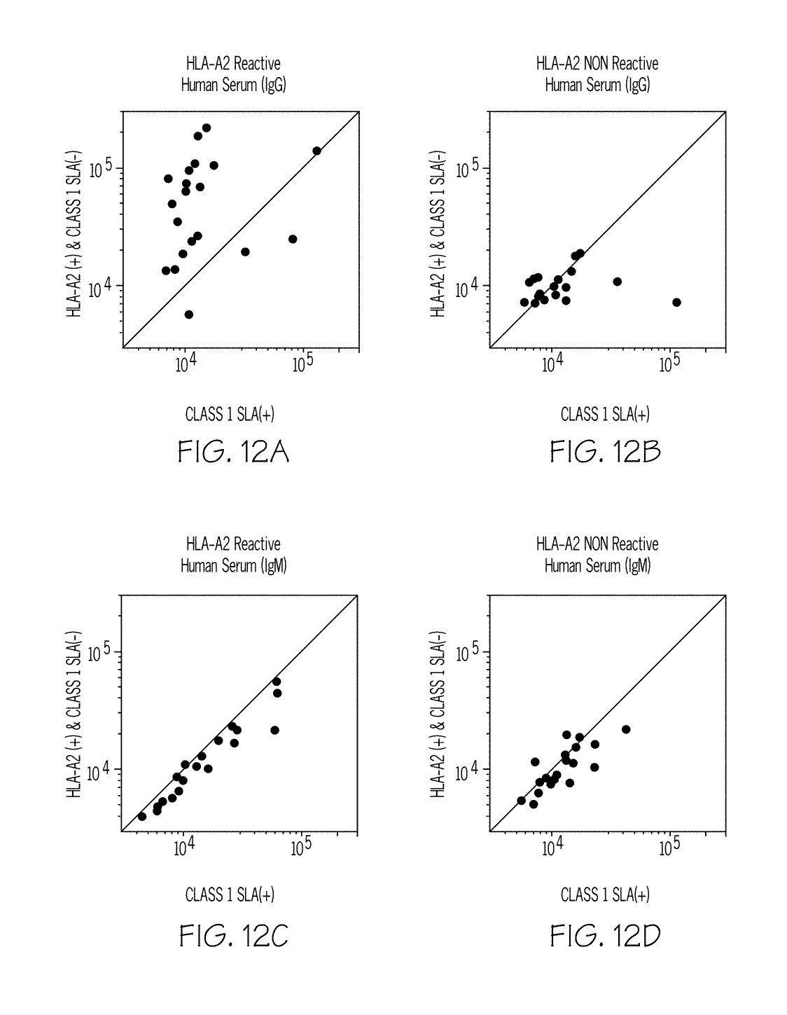

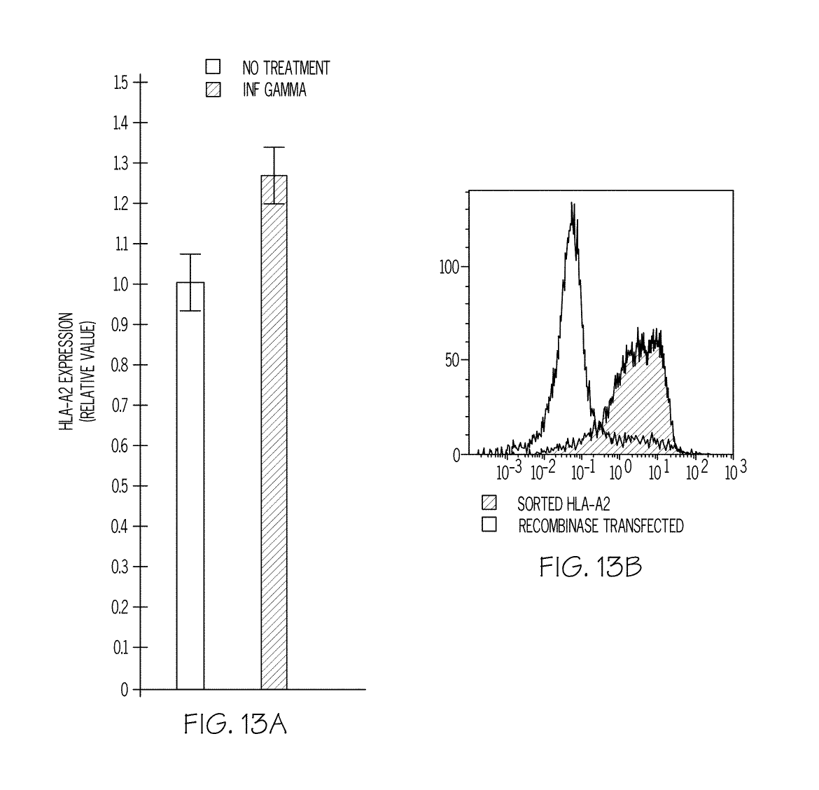

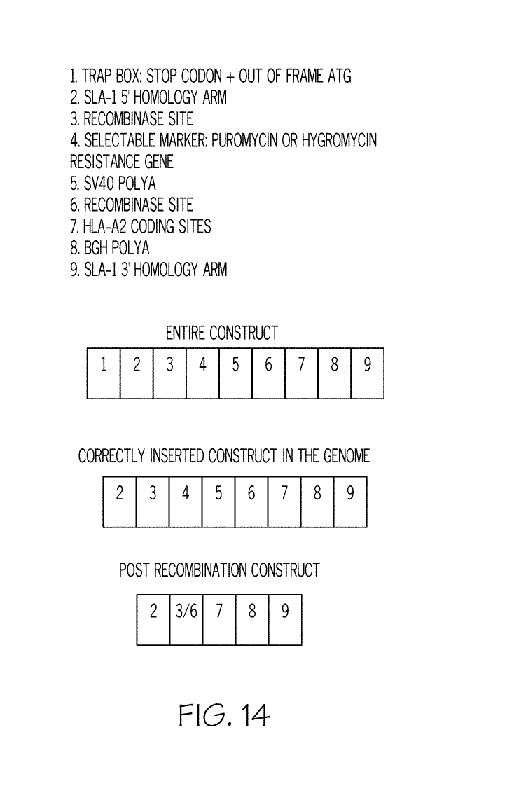

On Days 6-10 post-transfection, cells were harvested and counted using a hemacytometer. Cells were then resuspended at a density of two million cells per milliliter of PBS containing 0.5% w/v BSA and 0.1% v/v sodium azide. Cells were incubated on ice for 30 min prior to staining. Cells were then transferred to 5 mL polypropylene culture tubes and stained with either mouse IgG₁ FITC (AbD Serotec), SLA I FITC (AbD Serotec), mouse IgG₁ PE (Santa Cruz Biotechnology), or B2M PE (Santa Cruz Biotechnology) at a ratio of one microgram antibody to one million cells. Cells were stained for 30 minutes on ice, washed, and analyzed on an Accuri C6 cytometer (BD Biosciences). Event limits were set at 10,000 in forward and side scatter gate. Results of one such series of experiments are shown in FIG. 10. While not being limited by mechanism, B2microglobulin (B2M) is a partner to the porcine SLA class I molecule. The SLA class I molecule and B2M must associate for the SLA Class I allele to reach the cell surface. The absence of B2M binding indicates the absence of SLA Class I alleles that are not detectable by the anti-SLA class I antibody used in the studies.

The invention is not limited to the embodiments set forth herein for illustration but includes everything that is within the scope of the claims. Having described the invention with reference to the exemplary embodiments, it is to be understood that it is not intended that any limitations or elements describing the exemplary embodiments set forth herein are to be incorporated into the meanings of the patent claims unless such limitations or elements are explicitly listed in the claims. Likewise, it is to be understood that it is not necessary to meet any or all of the identified advantages or objects of the invention disclosed herein in order to fall within the scope of any claims, since the invention is defined by the claims, and since inherent and/or unforeseen advantages of the present invention may exist even though they may not be explicitly discussed herein.

Furthermore all references cited herein are hereby incorporated by reference in their entirety and for all purposes as if fully set forth herein.

1. A transgenic pig comprising a disrupted α(1,3)-galactosyltransferase, CMAH and SLA gene in the nuclear genome of at least one cell of said pig, wherein expression of α(1,3)-galactosyltransferase, CMAH and an SLA gene product is decreased as compared to a wild-type pig.

2. A porcine organ, tissue or cell isolated from said transgenic pig of claim 1.

3. (canceled)

4. The transgenic pig of claim 1 wherein when tissue from said pig is transplanted into a human, a rejection related symptom is improved as compared to when tissue from a wild-type pig is transplanted into a human.

5. The transgenic pig of claim 1 wherein when tissue from said pig is transplanted into a human, the rejection related symptom is selected from the group comprising a cellular rejection response related symptom, a humoral rejection response related symptom, a hyperacute rejection related symptom, an acute humoral xenograft reaction rejection related symptom and an acute vascular rejection response related symptom.

6. The transgenic pig of claim 1 wherein when a liver from said transgenic pig is exposed to human platelets, said liver exhibits reduced platelet uptake as compared to when a liver from a wild-type pig is exposed to human platelets.

7. A skin related product obtained from the transgenic pig of claim 1 wherein said skin related product exhibits reduced premature separation from a wound.

8. (canceled)

9. A method of preparing transplant material for xenotransplantation into a human, the method comprising providing the transgenic pig of claim 1 as a source of said transplant material and wherein said transplant material is selected from the group consisting of organs, tissues and cells, and wherein said transplant material has a reduced level of αGal antigens, a reduced level of Neu5GC antigens and a reduced level of SLA antigens.

10. A transgenic pig comprising a disrupted α(1,3)-galactosyltransferase, CMAH and SLA class I gene in the nuclear genome of at least one cell of said pig, wherein the disruption of said α(1,3)-galactosyltransferase gene is selected from the group of disruptions comprising a three base pair deletion adjacent to a G to A substitution, a single base pair deletion, a six base pair deletion, a two base pair insertion, a ten base pair deletion, five base pair deletion, a seven base pair deletion, an eight base pair substitution for a five base pair deletion, a single base pair insertion, a five base pair insertion, and both a five base pair deletion and a seven base pair deletion, wherein the disruption of said CMAH gene is selected from the group of disruptions comprising twelve base pair deletion, a five base pair substitution for a three base pair deletion, a four base pair insertion, a two base pair deletion, an eight base pair deletion, a five base pair deletion, a three base pair deletion, a two base pair insertion for a single base pair deletion, a twenty base pair deletion, a one base pair deletion, an eleven base pair deletion, wherein the disruption of said SLA class I gene is selected from the group of disruptions comprising a 276 base pair deletion, a 276 base pair deletion in exon 4, a 4 base pair deletion, a 4 base pair deletion in exon 4, a 2 base deletion, a 1 base pair insertion, and a frameshift mutation in exon 4 and wherein expression of α(1,3)-galactosyltransferase, CMAH and SLA is decreased as compared to a wild-type pig, and when tissue from said transgenic pig is transplanted into a human, a hyperacute rejection related symptom is improved as compared to when tissue from a wild-type pig is transplanted into a human.

11. A method of increasing the duration of the period between when a human subject is identified as a subject in need of a human liver transplant and when said human liver transplant occurs, said method comprising providing a liver from a transgenic pig comprising a disrupted α(1,3)-galactosyltransferase, CMAH and SLA gene in the nuclear genome of at least one cell of said pig, wherein expression of α(1,3)-galactosyltransferase, CMAH and SLA in said pig is decreased as compared to a wild-type pig, and surgically attaching said liver from said transgenic pig to said human subject in a therapeutically effective manner.

12. The method of claim 11, wherein said liver from said transgenic pig is surgically attached internal to said human subject.

13. (canceled)

14. The method of claim 11, wherein said liver is directly or indirectly attached to said subject.

15. A method of reducing premature separation of a skin related product from a human, comprising the steps of providing a transgenic pig comprising disrupted α(1,3)-galactosyltransferase, CMAH, and SLA genes wherein expression of α(1,3)-galactosyltransferase, CMAH and SLA in said pig is decreased as compared to a wild-type pig, and preparing a skin related product from said transgenic pig.

16. A method of improving a hyperacute rejection related symptom in a human subject comprising transplanting porcine transplant material having reduced levels of αGal antigens, reduced levels of Neu5GC antigens and reduced levels of SLA antigens into a subject in need of a transplant, wherein a hyperacute rejection related symptom is improved as compared to when porcine transplant material from a wild-type pig is transplanted into a human subject.

17. A cell culture reagent that exhibits an altered epitope profile wherein said cell culture reagent is isolated from a transgenic pig comprising disrupted α(1,3)-galactosyltransferase, CMAH, and SLA genes and wherein expression of α(1,3)-galactosyltransferase, CMAH and SLA in said transgenic pig is decreased as compared to a wild-type pig.

18. The cell culture reagent of claim 17, wherein said cell culture reagent is selected from the group comprising cell culture media, cell culture serum, cell culture additive and an isolated cell capable of proliferation.

19. The cell culture reagent of claim 17, wherein said cell culture reagent is isolated from a transgenic pig wherein the disruption of said α(1,3)-galactosyltransferase gene is selected from the group of disruptions comprising a three base pair deletion adjacent to a G to A substitution, a single base pair deletion, a six base pair deletion, a two base pair insertion, a ten base pair deletion, five base pair deletion, a seven base pair deletion, an eight base pair substitution for a five base pair deletion, a single base pair insertion, a five base pair insertion, and both a five base pair deletion and a seven base pair deletion, wherein the disruption of said CMAH gene is selected from the group of disruptions comprising twelve base pair deletion, a five base pair substitution for a three base pair deletion, a four base pair insertion, a two base pair deletion, an eight base pair deletion, a five base pair deletion, a three base pair deletion, a two base pair insertion for a single base pair deletion, a twenty base pair deletion, a one base pair deletion, an eleven base pair deletion, wherein the disruption of said SLA class I gene is selected from the group of disruptions comprising a 276 base pair deletion, a 276 base pair deletion in exon 4, a 4 base pair deletion, a 4 base pair deletion in exon 4, a 2 base deletion, a 1 base pair insertion, and a frameshift mutation in exon 4 and wherein expression of α(1,3)-galactosyltransferase, CMAH and SLA is decreased as compared to a wild-type pig.

20. A method of producing a compound of interest with an altered epitope profile, said method comprising the steps of providing a cell culture reagent that exhibits an altered epitope profile wherein said cell culture reagent is isolated from a transgenic pig comprising disrupted functional α(1,3)-galactosyltransferase, CMAH, and SLA genes and wherein expression of α(1,3)-galactosyltransferase, CMAH and SLA genes in said transgenic pig is decreased as compared to a wild-type pig, and incubating an isolated cell capable of expressing said compound of interest with said cell culture reagent; and wherein the level of Neu5Gc or alphaGal or SLA epitopes on said compound of interest is lower than the level of said epitopes on said compound of interest when said compound of interest is produced from an isolated cell incubated with a cell culture reagent isolated from a wild-type pig.

21. The method of claim 20, wherein said compound of interest is selected from the group comprising glycolipids and glycoproteins.

22. (canceled)

23. The method of claim 20 wherein said cell culture reagent is isolated from a transgenic pig wherein the disruption of said α(1,3)-galactosyltransferase gene is selected from the group of disruptions comprising a three base pair deletion adjacent to a G to A substitution, a single base pair deletion, a six base pair deletion, a two base pair insertion, a ten base pair deletion, five base pair deletion, a seven base pair deletion, an eight base pair substitution for a five base pair deletion, a single base pair insertion, a five base pair insertion, and both a five base pair deletion and a seven base pair deletion, wherein the disruption of said CMAH gene is selected from the group of disruptions comprising twelve base pair deletion, a five base pair substitution for a three base pair deletion, a four base pair insertion, a two base pair deletion, an eight base pair deletion, a five base pair deletion, a three base pair deletion, a two base pair insertion for a single base pair deletion, a twenty base pair deletion, a one base pair deletion, an eleven base pair deletion, wherein the disruption of said SLA class I gene is selected from the group of disruptions comprising a 276 base pair deletion, a 276 base pair deletion in exon 4, a 4 base pair deletion, a 4 base pair deletion in exon 4, a 2 base deletion, a 1 base pair insertion, and a frameshift mutation in exon 4 and wherein expression of α(1,3)-galactosyltransferase, CMAH and SLA is decreased as compared to a wild-type pig.

24. A porcine transplant material for transplantation into a human, wherein lipids and proteins of said transplant material have a reduced level of αGal antigens, Neu5Gc antigens and SLA antigens.

25. A transgenic pig comprising a disrupted α(1,3)-galactosyltransferase and SLA class I gene in the nuclear genome of at least one cell of said pig, wherein expression of α(1,3)-galactosyltransferase and an SLA gene product is decreased as compared to a wild-type pig.

26. The transgenic pig of claim 25 wherein the disruption of said SLA class I gene is selected from the group comprising exon 4 disruptions, a 276 base pair deletion, a 276 base pair deletion in exon 4, a 4 base pair frameshift mutation, a two base pair deletion and a one base pair insertion.

27. The transgenic pig of claim 25 wherein the disruption of said α(1,3)-galactosyltransferase gene is selected from the group comprising a three base pair deletion adjacent to a G to A substitution, a single base pair deletion, a six base pair deletion, a two base pair insertion, a ten base pair deletion, five base pair deletion, a seven base pair deletion, an eight base pair substitution for a five base pair deletion, a single base pair insertion, a five base pair insertion, and both a five base pair deletion and a seven base pair deletion.

28. A transgenic pig comprising a nucleotide sequence encoding a class I HLA polypeptide in the nuclear genome of at least one cell of said pig, wherein expression of said HLA polypeptide is increased as compared to a wild-type pig and further comprising a disrupted α(1,3)-galactosyltransferase and SLA gene in the nuclear genome of at least one cell of said pig, wherein expression of α(1,3)-galactosyltransferase and an SLA gene product is decreased as compared to a wild-type pig.

29. The transgenic pig of claim 28 further comprising a disrupted CMAH gene in the nuclear genome of at least one cell of said pig, wherein expression of CMAH is decreased as compared to a wild-type pig.

30. The transgenic pig of claim 28, wherein said nucleotide sequence encodes a class I HLA polypeptide selected from the group comprising HLA-A, HLA-B, HLA-C, HLA-C and HLA-A2.

31. A porcine organ, tissue or cell isolated from said transgenic pig of claim 28.

32. (canceled)

33. The transgenic pig of claim 28 wherein when tissue from said pig is transplanted into a human, a rejection related symptom is improved as compared to when tissue from a wild-type pig is transplanted into a human.

34. (canceled)

35. The transgenic pig of claim 28 wherein when a liver from said transgenic pig is exposed to human platelets, said liver exhibits reduced platelet uptake as compared to when a liver from a wild-type pig is exposed to human platelets.

36. A skin related product obtained from the transgenic pig of claim 28 wherein said skin related product exhibits reduced premature separation from a wound.

37. (canceled)

38. A method of preparing transplant material for xenotransplantation into a human, the method comprising providing the transgenic pig of claim 28 as a source of said transplant material and wherein said transplant material is selected from the group consisting of organs, tissues and cells, and wherein said transplant material has an increased level of class I HLA polypeptides and reduced level of αGal antigens, and a reduced level of SLA antigens.

39. A method of preparing transplant material for xenotransplant into a human, the method comprising providing the transgenic pig of claim 29 as a source of said transplant material and wherein said transplant material is selected from the group consisting of organs, tissues and cells, and wherein said transplant material has an increased level of class I HLA polypeptides and reduced level of αGal antigens, a reduced level of Neu5Gc antigens and a reduced level of SLA antigens.

40. The transgenic pig of claim 28, wherein the disruption of said α(1,3)-galactosyltransferase gene is selected from the group of disruptions comprising a three base pair deletion adjacent to a G to A substitution, a single base pair deletion, a six base pair deletion, a two base pair insertion, a ten base pair deletion, five base pair deletion, a seven base pair deletion, an eight base pair substitution for a five base pair deletion, a single base pair insertion, a five base pair insertion, and both a five base pair deletion and a seven base pair deletion, wherein the disruption of said SLA class I gene is selected from the group of disruptions comprising an insertion, a 276 base pair deletion, a 276 base pair deletion in exon 4, a 4 base pair deletion, a 4 base pair deletion in exon 4, a 2 base deletion, a 1 base pair insertion, and a frameshift mutation in exon 4 and wherein expression of α(1,3)-galactosyltransferase and SLA is decreased as compared to a wild-type pig, and when tissue from said transgenic pig is transplanted into a human, a hyperacute rejection related symptom is improved as compared to when tissue from a wild-type pig is transplanted into a human.

41. The transgenic pig of claim 29 wherein the disruption of said CMAH gene is selected from the group of disruptions comprising twelve base pair deletion, a five base pair substitution for a three base pair deletion, a four base pair insertion, a two base pair deletion, an eight base pair deletion, a five base pair deletion, a three base pair deletion, a two base pair insertion for a single base pair deletion, a twenty base pair deletion, a one base pair deletion, an eleven base pair deletion, and wherein expression of CMAH is decreased as compared to wildtype pig, and when tissue from said transgenic pig is transplanted into a human, a hyperacute rejection related symptom is improved as compared to when tissue from a wild-type pig is transplanted into a human.

42. A method of increasing the duration of the period between when a human subject is identified as a subject in need of a human liver transplant and when said human liver transplant occurs, said method comprising providing a liver from a transgenic pig comprising a nucleotide sequence encoding a class I HLA polypeptide in the nuclear genome of at least one cell of said pig, wherein expression of said HLA polypeptide is increased as compared to a wild-type pig and further comprising a disrupted α(1,3)-galactosyltransferase, CMAH and SLA gene in the nuclear genome of at least one cell of said pig, wherein expression of α(1,3)-galactosyltransferase, CMAH and SLA in said pig is decreased as compared to a wild-type pig, and surgically attaching said liver from said transgenic pig to said human subject in a therapeutically effective manner.

43. The method of claim 42, wherein said liver from said transgenic pig is surgically attached internal to said human subject.

44. (canceled)

45. The method of claim 42, wherein said liver is directly or indirectly attached to said subject.

46. A method of reducing premature separation of a skin related product from a human, comprising the steps of providing a transgenic pig comprising a nucleotide sequence encoding a class I HLA polypeptide in the nuclear genome of at least one cell of said pig, wherein expression of said HLA polypeptide is increased as compared to a wild-type pig and further comprising disrupted α(1,3)-galactosyltransferase, CMAH, and SLA genes wherein expression of α(1,3)-galactosyltransferase, CMAH and SLA in said pig is decreased as compared to a wild-type pig, and preparing a skin related product from said transgenic pig.

47. A method of improving a hyperacute rejection related symptom in a human subject comprising transplanting porcine transplant material having increased levels of a class I HLA polypeptide and reduced levels of αGal antigens, reduced levels of Neu5GC antigens and reduced levels of SLA antigens into a subject in need of a transplant, wherein a hyperacute rejection related symptom is improved as compared to when porcine transplant material from a wild-type pig is transplanted into a human subject.

48. A cell culture reagent that exhibits an altered epitope profile wherein said cell culture reagent is isolated from a transgenic pig comprising a nucleotide sequence encoding a class I HLA polypeptide in the nuclear genome of at least one cell of said pig, wherein expression of said HLA polypeptide is increased as compared to a wild-type pig and further comprising disrupted α(1,3)-galactosyltransferase, CMAH, and SLA genes and wherein expression of α(1,3)-galactosyltransferase, CMAH and SLA in said transgenic pig is decreased as compared to a wild-type pig.

49. (canceled)

50. The cell culture reagent of claim 48, wherein said cell culture reagent is isolated from a transgenic pig wherein the disruption of said α(1,3)-galactosyltransferase gene is selected from the group of disruptions comprising a three base pair deletion adjacent to a G to A substitution, a single base pair deletion, a six base pair deletion, a two base pair insertion, a ten base pair deletion, five base pair deletion, a seven base pair deletion, an eight base pair substitution for a five base pair deletion, a single base pair insertion, a five base pair insertion, and both a five base pair deletion and a seven base pair deletion, wherein the disruption of said CMAH gene is selected from the group of disruptions comprising twelve base pair deletion, a five base pair substitution for a three base pair deletion, a four base pair insertion, a two base pair deletion, an eight base pair deletion, a five base pair deletion, a three base pair deletion, a two base pair insertion for a single base pair deletion, a twenty base pair deletion, a one base pair deletion, an eleven base pair deletion, wherein the disruption of said SLA class I gene is selected from the group of disruptions comprising an insertion, a 276 base pair deletion, a 276 base pair deletion in exon 4, a 4 base pair deletion, a 4 base pair deletion in exon 4, a 2 base deletion, a 1 base pair insertion, and a frameshift mutation in exon 4 and wherein expression of α(1,3)-galactosyltransferase, CMAH and SLA is decreased as compared to a wild-type pig.

51. A method of producing a compound of interest with an altered epitope profile, said method comprising the steps of providing a cell culture reagent of claim 40; and wherein the level of Neu5Gc or alphaGal or SLA epitopes on said compound of interest is lower than the level of said epitopes on said compound of interest when said compound of interest is produced from an isolated cell incubated with a cell culture reagent isolated from a wild-type pig.

52. The method of claim 51, wherein said compound of interest is selected from the group comprising glycolipids and glycoproteins.

53.-55. (canceled)
